(19) United States
(12) Patent Application Publication
(10) Pub. No.: US 2023/0257337 A1
(43) Pub. Date: Aug. 17, 2023

(54) METHOD FOR PRODUCING POLYGYLCEROL ESTERS OF POLYCARBOXYLIC ACIDS ESTERIFIED WITH OXOBUTANOL

(71) Applicant: IOI Oleo GmbH, Witten (DE)

(72) Inventors: Dirk LOCHMANN, Witten (DE); Sebastian REYER, Witten (DE); Michael STEHR, Witten (DE)

(21) Appl. No.: 18/004,753

(22) PCT Filed: Sep. 7, 2020
(86) PCT No.: PCT/EP2020/074892

§ 371 Date: Jan. 9, 2023

(30) Foreign Application Priority Data
Jul. 13, 2020 (WO) PCT/EP2020/069712

(51) Int. Cl. C07C 67/08; C07C 55/24; A61P 3/00; C12P 7/62; A23L 33/10

(52) U.S. Cl. CPC C07C 67/08; C07C 55/24; A61P 3/00; C12P 7/62; A23L 33/10

(57) ABSTRACT

The invention relates to a method for producing polyglycerol esters of polycarboxylic acids esterified with oxobutanol, especially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, as well as the products thus obtained and their use.

### CROSS-REFERENCES TO RELATED APPLICATIONS

This application is a National Stage filing of International Application PCT/EP 2020/074892 filed Sep. 7, 2020, entitled “Method for Producing Polyglycerol Esters of Polycarboxylic Acids Esterified with Oxobutanol” claiming priority to PCT/EP 2020/069712 filed Jul. 13, 2020. The subject application claims priority to PCT/EP 2020/074892 and PCT/EP 2020/069712 and incorporates all by reference herein, in their entirety.

### BACKGROUND OF THE INVENTION

The present invention relates to the field of keto bodies and related metabolism and the therapy of related diseases.

Especially, the present invention relates to a method for producing polyglycerol esters of polycarboxylic acids esterified with oxobutanol, as well as the reaction products thus obtainable or thus prepared (i.e. polyglycerol esters of polycarboxylic acids esterified with oxobutanol) and their use, especially in pharmaceutical compositions, such as drugs or medicaments, or in food and/or food products, as well as their further applications or uses.

Furthermore, the present invention relates to pharmaceutical compositions, especially drugs or medicaments, comprising the reaction products (i.e. polyglycerol esters of polycarboxylic acids esterified with oxobutanol) obtainable or produced according to the inventive method, as well as their applications or uses.

Finally, the present invention relates to food and/or food products, especially food supplements, functional foods, novel foods, food additives, food supplements, dietary foods, power snacks, appetite suppressants and strength and/or endurance sports supplements, which comprise the reaction products (i.e. polyglycerol esters of polycarboxylic acids esterified with oxobutanol) obtainable or produced according to the inventive method, as well as their applications or uses.

In the human energy metabolism, glucose is the short-term available energy carrier, which is metabolized into energy in the mitochondria by releasing water and carbon dioxide. The glycogen stores of the liver are already emptied during the sleep period during the night. However, especially the human central nervous system (CNS) and the heart require a permanent energy supply.

The physiological alternative to glucose, which is mainly available to the central nervous system, are the so-called keto bodies (synonymously also called ketone bodies).

The term keto body is especially a collective term for three compounds, which are formed mainly in catabolic metabolic states (such as hunger, reduction diets or low-carbohydrate diets) and may lead to ketosis. The term keto bodies includes especially the three compounds acetoacetate (synonymously also referred to as acetoacetate) and acetone as well as 3-hydroxybutyric acid (hereinafter also synonymously referred to as beta-hydroxybutyric acid or BHB or 3-BHB) or its salt (i.e. 3-hydroxybutyrate or beta-hydroxybutyrate), the latter being the most important of the three aforementioned compounds. 3-Hydroxybutyric acid or its salt occurs physiologically as the (R)-enantiomer, i.e. as (R)-3-hydroxybutyric acid (synonymously also called (3R)-3-hydroxybutyric acid to emphasize the center of chirality in the 3-position) or its salt.

These keto bodies are also provided physiologically in large amounts from lipids stored in the body by lipolysis during fasting or starvation and replace the energy source glucose almost completely.

The keto bodies are formed in the liver from acetyl coenzyme A (=acetyl-CoA), which originates from beta-oxidation; they represent a transportable form of the acetyl coenzyme A in the human body. However, in order to utilize the keto bodies, the brain and muscles must first adapt by expressing enzymes that are required to convert keto bodies back into acetyl coenzyme A. Especially in times of hunger, the keto bodies contribute a considerable amount to energy production. For example, after some time the brain is able to get by with only a third of the daily amount of glucose.

Physiologically, the keto bodies are synthesized from two molecules of activated acetic acid in the form of acetyl coenzyme A, the normal intermediate product of fatty acid degradation, which is extended using a further acetyl coenzyme A unit and the enzyme HMG-CoA-synthase to the intermediate product 3-hydroxy-3-methyl-glutaryl-CoA (HMG-CoA), wherein finally the HMG-CoA-lyase cleaves off the acetoacetate. These three steps take place exclusively in the mitochondria of the liver (lynen cycle), wherein 3-hydroxybutyrate is finally formed in the cytosol by the D-beta-hydroxybutyrate dehydrogenase. HMG-CoA is also an end product of the degradation of the amino acid leucine, while acetoacetate is formed during the degradation of the amino acids phenylalanine and tyrosine.

Spontaneous decarboxylation turns acetoacetate into acetone; it can occasionally be perceived in the breath of diabetics and dieters. It cannot be further used by the body. However, the proportion of acetone in the keto bodies is small.

Acetoacetate is thus reductively converted into the physiologically relevant form of 3-hydroxybutyric acid or 3-hydroxybutyrate, but can also decompose into the physiologically unusable acetone with the release of carbon dioxide, which is detectable and olfactory perceptible in severe ketosis, a ketoacidosis (e. g. in diabetes mellitus type 1 patients without insulin substitution), in the urine and in the exhaled air.

3-Hydroxybutyric acid is currently used and marketed in the weight training sector as a sodium, magnesium or calcium salt.

However, 3-hydroxybutyric acid is not known or only in very small quantities to humans in evolutionary terms, since plants do not produce 3-hydroxybutyric acid and 3-hydroxybutyric acid in the animal organism only occurs in dead emaciated animals in ketosis, so that 3-hydroxybutyric acid causes nausea when administered orally. 3-Hydroxybutyric acid in the form of free acid and its salts also taste very bitter and can cause severe vomiting and nausea.

Moreover, patients, especially newborns, but also adults cannot permanently tolerate large amounts of salts of 3-hydroxybutyric acid, as these compounds can have a kidney-damaging effect.

In addition, the plasma half-life of 3-hydroxybutyric acid and its salts is so short that even if several grams are taken, the ketosis lasts only for about three to four hours, i.e. patients cannot benefit continuously from a therapy with 3-hydroxybutyric acid or its salts, especially at night. In case of metabolic diseases this can lead to life-threatening situations.

Therefore, in the case of the therapy of such metabolic diseases, so-called medium-chain triglycerides, so-called MCTs, are currently used for ketogenic therapy, i.e. the metabolic conversion of caproic, caprylic and capric acid (i.e. of saturated linear C₆-, C₈- and C₁₀-fatty acids) from the corresponding triglycerides is intended.

Basically, however, from a pharmaceutical and clinical point of view, 3-hydroxybutyric acid is a more effective pharmaceutical-pharmacological target molecule, which, according to the prior art, could in principle be used for the therapy of a large number of diseases, but cannot be used due to its lack of physiological compatibility (e. g. in diseases in connection with a malfunction of the energy metabolism, especially keto-body metabolism, or neurodegenerative diseases such as dementia, Alzheimer's disease, Parkinson's disease, etc., lipometabolic diseases etc.).

The following table illustrates purely exemplary, but by no means limiting, potential therapy options or possible indications for the active ingredient 3-hydroxybutyric acid.

| Indication | Therapeutic effect |
|---|---|
| Traumatic brain injury | Under BHB the apoptosis and necrosis rate of nerve cells decreases. |
| Stroke | Under BHB the apoptosis and necrosis rate of nerve cells decreases. |
| Refeeding syndrome | In case of anorexia, discontinuation of enteral or parenteral nutrition |
| | and after long periods of hunger, the consumption of starch or |
| | glucose can lead to death (see also WHO scheme peanut paste). BHB |
| | can be used here as a therapeutic agent to achieve normal food intake |
| Appetite suppressant | BHB suppresses the feeling of hunger in the central nervous system |
| | (CNS). |
| Epilepsy | Conventional ketogenic diet to significantly reduce the frequency of |
| | seizures has extremely poor patient tolerance. BHB offers an |
| | immediately effective alternative here. |
| Alzheimer's disease, | Under BHB patients show better cognitive performance, BHB is also |
| dementia | effective in the prevention of neurodegenerative diseases. |
| Disorders of fatty acid | Compensation of a nutrient deficiency in case of defect in energy |
| oxidation (e.g. electron | metabolism. |
| transfer protein defect) | |

Therefore, it is desirable from a pharmaceutical and clinical point of view to be able to find effective precursors or metabolites which physiologically allow direct or indirect access to 3-hydroxybutyric acid or its salts, especially in the physiological metabolism of the human or animal body.

Consequently, the prior art has not lacked attempts to find physiologically suitable precursors or metabolites for 3-hydroxybutyric acid or its salts. So far, however, no efficient compounds have been found in the prior art. Also, access to such compounds is not or not readily possible according to the prior art.

### BRIEF SUMMARY OF THE INVENTION

The problem underlying the present invention is thus the provision of an efficient method for producing physiologically suitable or physiologically compatible precursors and/or metabolites of 3-hydroxybutyric acid (i.e. beta-hydroxybutyric acid or BHB or 3-BHB) or their salts.

Such method should especially make the respective BHB precursors and/or BHB metabolites accessible in an efficient way, especially in larger quantities and without significant amounts of toxic by-products.

In a completely surprising way, the applicant has now discovered that polyglycerol esters of polycarboxylic acids esterified with oxobutanol represent an efficient and physiologically effective or physiologically compatible precursor and/or metabolite for the keto body 3-hydroxybutyric acid or its salts and has in this context been able to find or develop an efficient method for producing these compounds, which allows direct and effective, especially economic as well as industrially feasible access to these compounds.

To solve the problem described above, the present invention therefore proposes—according to a first aspect of the present invention—a method for producing polyglycerol esters of polycarboxylic acids esterified with oxobutanol according to the teaching herein; further, especially special and/or advantageous embodiments of the inventive method are the subject-matter of the relevant [[sub]]claims.

Furthermore, the present invention relates—according to a second aspect of the present invention—to a reaction intermediate product or a polycarboxylic acid ester of oxobutanol obtainable according to the inventive method or a respective mixture of at least two different polycarboxylic acid esters of oxobutanol, and furthermore to a reaction product or a polyglycerol ester of polycarboxylic acids esterified with oxobutanol obtainable according to the inventive method or a respective mixture of at least two polyglycerol esters of polycarboxylic acids esterified with oxobutanol; further, especially special and/or advantageous embodiments of this aspect of the invention are the subject-matter of the relevant subclaims.

Likewise, the present invention—according to a third aspect of the present invention—relates to a pharmaceutical composition, especially a drug or medicament, especially special and/or advantageous embodiments of this aspect of the invention.

Furthermore, the present invention—according to a fourth aspect of the present invention—relates to an inventive reaction product or an inventive polyglycerol ester of polycarboxylic acids esterified with oxobutanol or an inventive mixture according of at least two polyglycerol esters of polycarboxylic acids esterified with oxobutanol for the prophylactic and/or therapeutic treatment or for use in the prophylactic and/or therapeutic treatment of diseases of the human or animal body.

Furthermore, the present invention—according to a fifth aspect of the present invention—relates to the use of an inventive reaction product or an inventive polyglycerol ester of polycarboxylic acids esterified with oxobutanol or an inventive mixture of at least two polyglycerol esters of polycarboxylic acids esterified with oxobutanol for the prophylactic and/or therapeutic treatment or for producing a medicament for the prophylactic and/or therapeutic treatment of diseases of the human or animal body.

Furthermore, the present invention—according to a sixth aspect of the present invention—relates to the use of an inventive reaction product or an inventive polyglycerol ester of polycarboxylic acids esterified with oxobutanol or an inventive mixture of at least two polyglycerol esters of polycarboxylic acids esterified with oxobutanol.

Furthermore, the present invention—according to a seventh aspect of the present invention—relates to a food and/or food product; further, especially special and/or advantageous embodiments of the food and/or food product.

Finally, the present invention—according to an eighth aspect of the present invention—relates to the use of an inventive reaction product or an inventive polyglycerol ester of polycarboxylic acids esterified with oxobutanol or an inventive mixture of at least two polyglycerol esters of polycarboxylic acids esterified with oxobutanol in a food and/or a food product.

It goes without saying that following features, embodiments, advantages and the like, which are subsequently listed below only with regard to one aspect of the invention for the purpose of avoiding repetition, naturally also apply accordingly to the other aspects of the invention, without this requiring a separate mention.

Furthermore, it goes without saying that individual aspects and embodiments of the present invention are also considered disclosed in any combination with other aspects and embodiments of the present invention and, especially, any combination of features and embodiments, as it results from back references of all patent claims, is also considered extensively disclosed with regard to all resulting combination possibilities.

With respect to all relative or percentage weight-based data provided below, especially relative quantity or weight data, it should further be noted that within the scope of the present invention these are to be selected by the person skilled in the art such that they always add up to 100% or 100% by weight, respectively, including all components or ingredients, especially as defined below; however, this is self-evident for the person skilled in the art.

In addition, the skilled person may, if necessary, deviate from the following range specifications without leaving the scope of the present invention.

Additionally, it applies that all values or parameters or the like specified in the following can be determined or identified in principle with standardized or explicitly specified determination methods or otherwise with the determination or measurement methods that are otherwise familiar to a person skilled in the art.

Having stated this, the present invention will be described in more detail hereinafter:

### DETAILED DESCRIPTION OF THE INVENTION

The subject-matter of the present invention—according to a first aspect of the present invention—is thus a method for producing polyglycerol esters of polycarboxylic acids esterified with oxobutanol, especially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol,

wherein
(i) at least one oxobutanol, especially 3-hydroxybutanoate, preferentially 4-oxo-2-butanol, preferably 4-oxo-4-(C₁-C₅-alkoxy)-2-butanol or 4-oxo-4-(hydroxy-C₃-C₅-alkoxy)-2-butanol, of the general formula (I)

CH₃—CH(OH)—CH₂—C(O)OR¹ (I)
wherein, in the general formula (I), the radical R¹ represents C₁-C₃-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl,
(ii) at least one polycarboxylic acid (II), especially at least one polycarboxylic acid comprising at least two carboxyl groups, and
(iii) at least one polyglycerol (III)

are reacted and/or caused to react with one another,

so that, as a reaction product (IV), one or more polyglycerol esters of polycarboxylic acids esterified with oxobutanol, especially one or more polyglycerol esters of polycarboxylic acids esterified with 4-oxo-2-butanol, preferentially one or more mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, are obtained.

According to the present invention, there is thus provided especially a production method for polyglycerol esters of polycarboxylic acids esterified with 3-hydroxybutanoate. 3-Hydroxybutanoate may also be referred to synonymously as 3-hydroxybutyric acid ester or else as a 4-oxo-2-butanol.

Strictly speaking, oxobutanol or 3-hydroxybutanoate of the general formula (I) is either a (C₁-C₅-alkyl)-3-hydroxybutanoate (=3-hydroxybutyric acid (C₁-C₅-alkyl) ester), i.e. a C₁-C₅-alkyl ester of 3-hydroxybutyric acid, which may also be referred to synonymously as a 4-oxo-4-(C₁-C₅-alkoxy)-2-butanol, or else a (hydroxy-C₃-C₅-alkyl)-3-hydroxybutanoate (=3-hydroxybutyric acid (hydroxy-C₃-C₅-alkyl)ester), i.e. a hydroxy-C₃-C₅-alkyl ester of 3-hydroxybutyric acid, which may also be referred to synonymously as a 4-oxo-4-(hydroxy-C₃-C₅-alkoxy)-2-butanol.

In this context, the 3-hydroxybutanoates can be prepared by esterification of the free 3-hydroxybutyric acid with the corresponding alcohols (e.g. monoalcohols such as ethanol etc. or diols). The synthesis of hydroxybutyl 3-hydroxybutanoate (i.e. 3-hydroxybutanoate of the general formula (I) with R¹=hydroxybutyl) is shown schematically below:

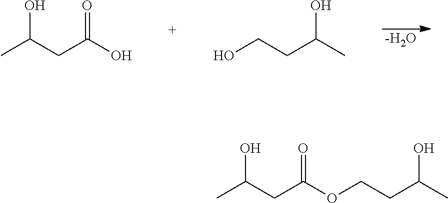

Furthermore, the synthesis of hydroxypentyl 3-hydroxybutanoate (i.e. 3-hydroxybutanoate of the general formula (I) with R¹=hydroxypentyl) is shown schematically below:

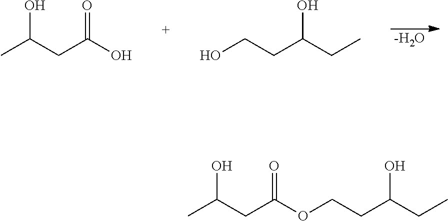

The production or synthesis of further 3-hydroxybutanoates proceeds analogously.

In the inventive method, the starting material or compound 3-hydroxybutanoate of the general formula (I) acts as an esterification alcohol via the hydroxyl group in the 3-position and reacts with the carboxyl group of the polycarboxylic acid (II) so that, as the reaction intermediate product (IV′), a corresponding polycarboxylic acid ester of 3-hydroxybutanoate, i.e. a corresponding polycarboxylic acid ester of 4-oxo-4-(C₁-C₅-alkoxy)-2-butanol or of 4-oxo-4-(hydroxy-C₃-C₅-alkoxy)-2-butanol, is formed.

In the context of the present invention, the polycarboxylic acid (II) used is an organic polycarboxylic acid; i.e. the polycarboxylic acid (II) is an organic compound with multiple carboxyl groups (—COOH), which consequently has an acidic character.

The polyglycerol esters obtained according to the invention are esters of polyglycerol (such as diglycerol) and precisely not polyesterified glycerol (i.e. especially n mono-, di- and triglycerides, i.e. the mono-, di- and tri-valent esters of glycerol and 1,2,3-propanetriol, respectively).

Thus, in the context of the present invention, monovalence and polyvalence refer to the number of esterified groups of the polyglycerol (i.e. groups esterified with polycarboxylic acid esters of oxobutanol).

Surprisingly, the applicant has found an efficient as well as effective way to provide 3-hydroxybutyric acid or its derivative in a physiologically as well as organoleptically compatible form, wherein the 3-hydroxybutyric acid can still be readily released, especially by the animal or human body.

Furthermore, the applicant has succeeded in providing the organoleptically and physiologically compatible form of 3-hydroxybutyric acid in such a way that a retardation effect is present; i.e. the 3-hydroxybutyric acid is released continuously over a longer period of time, especially by the human or animal body.

In addition, the other degradation or cleavage products (i.e. the cleavage products released in addition to 3-hydroxybutyric acid) can be utilized by the body, or at least processed by the body. Especially, cleavage products are released which are reactants, products or intermediates of the citrate cycle or are derivatives or salts formed by oxidation of a reactant, product or intermediate of the citrate cycle. Thus, the further degradation or cleavage products formed during the release of 3-hydroxybutyric acid can also be used as an energy source by the animal or human body. These cleavage products are typically the polycarboxylic acid (II). In addition, polyglycerol is a non-toxic and physiologically compatible carrier that is readily excreted by the body.

By using polyglycerol, which has a large number of hydroxyl groups, it is possible to provide a molecule with a high density of active ingredients, especially a high density of 3-hydroxybutanoates or 3-BHB or corresponding derivatives. Furthermore, in this context, a high density can also be provided of polycarboxylic acids, which—as previously stated—can be used as a further energy source by the animal and/or human body.

As stated above, the applicant has, quite surprisingly, discovered that the polyglycerol esters of polycarboxylic acids esterified with oxobutanol, especially polyglycerol esters of polycarboxylic acids esterified with 4-oxo-2-butanol, preferentially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, thus produced are efficient, since physiologically compatible and also organoleptically compatible precursors and/or metabolites of 3-hydroxybutyric acid or their salts, which can also be used in larger quantities in pharmaceutical or clinical applications.

The above-mentioned polyglycerol esters of polycarboxylic acids esterified with oxobutanol, especially polyglycerol esters of polycarboxylic acids esterified with 4-oxo-2-butanol, preferentially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, which are accessible for the first time in an efficient manner through the production method according to the invention, thus represent a physiologically and pharmacologically relevant alternative to free 3-hydroxybutyric acid or its salts and are moreover organoleptically compatible.

The production of polyglycerol esters of polycarboxylic acids esterified with oxobutanol, especially polyglycerol esters of polycarboxylic acids esterified with 4-oxo-2-butanol, preferentially mono- or polyvalent polyglycerol esters of polycarboxylic acids esterified with 4-oxo-4-(C₁-C₅-alkoxy)-2-butanol or with 4-oxo-4-(hydroxy-C₁-C₅-alkoxy)-2-butanol, by means of conventional organic synthesis is complex and costly, since 3-hydroxybutyric acid as well as its salts and esters have an increased tendency to polymerize and to undergo other undesirable side reactions (e. g. dehydration, decomposition, etc.). Within the scope of the present invention, it was possible for the first time to provide an efficiently working production method with which polyglycerol esters of polycarboxylic acids esterified with oxobutanol, especially polyglycerol esters of polycarboxylic acids esterified with 4-oxo-2-butanol, preferentially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, can be produced without undesired side reactions.

The inventive method thus makes it possible for the first time to provide non-toxic polyglycerol esters of polycarboxylic acids esterified with oxobutanol, especially polyglycerol esters of polycarboxylic acids esterified with 4-oxo-2-butanol, preferentially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, from known, commercially available and above all physiologically harmless components or educts (starting compounds). The resulting polyglycerol esters of polycarboxylic acids esterified with oxobutanol, especially polyglycerol esters of polycarboxylic acids esterified with 4-oxo-2-butanol, preferentially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, can be broken down physiologically, especially in the stomach and/or bowl, and release or generate the target molecule “3-hydroxybutyric acid” or its salts or esters as active ingredient or active component.

In addition, the aforementioned polyglycerol esters of polycarboxylic acids esterified with oxobutanol, especially polyglycerol esters of polycarboxylic acids esterified with 4-oxo-2-butanol, preferentially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, also comprise an acceptable taste to ensure compatibility even when administered orally in larger quantities over a longer period of time (e. g. administration of 50 g daily dose or more).

Similarly, the production method according to the invention makes it possible to provide the polyglycerol esters of polycarboxylic acids esterified with oxobutanol, especially polyglycerol esters of polycarboxylic acids esterified with 4-oxo-2-butanol, preferentially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, free from toxic impurities.

In addition, with appropriate starting materials, the method can also be carried out enantioselectively. For example, according to the invention, the production method allows the biologically relevant form, i.e. the (R)-enantiomer of BHB, to be enriched, for example by enzyme catalysis or the targeted selection of the starting materials (reactants), as not to burden the renal system of patients when administered orally (i.e. elimination via the kidneys). In principle, however, it is also possible, and under certain conditions may be useful, to enrich the (S)-enantiomer of BHB.

In addition, the inventive production method, including optional further processing or purification steps, can be operated economically and can also be implemented on a large scale.

Especially, the inventive production method uses commercially available starting compounds or starting compounds, which can be synthesized by simple methods that can be carried out on a large scale, and furthermore allows a relatively simple process management even in case of large-scale implementation.

In contrast to conventional prior art production methods, the production method according to the invention does not use complex starting materials and does not use protective groups. Nevertheless, excellent yields are achieved in accordance with the invention, wherein the formation of by-products is minimized or avoided.

In addition, the inventive method is simple and economical. Especially, the method according to the invention is usually carried out in the absence of solvents and/or without any solvent (i.e. as a reaction in mass or as a reaction in substance or as a so-called bulk reaction); consequently, the reaction products obtained are not contaminated with solvent and no solvent has to be removed and disposed of or recycled in a costly and energy-intensive manner after the method or reaction has been carried out. Furthermore, no toxic by-products are formed.

The production method according to the invention usually results in a mixture of different polyglycerol esters of polycarboxylic acids esterified with oxobutanol, especially polyglycerol esters of polycarboxylic acids esterified with 4-oxo-2-butanol, preferentially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, i.e. in a mixture of at least two different polyglycerol esters of polycarboxylic acids esterified with oxobutanol, especially polyglycerol esters of polycarboxylic acids esterified with 4-oxo-2-butanol, preferentially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol. The resulting raw reaction product or raw mixture can be purified by known methods, especially by removing any remaining starting compounds and/or any by-products present, and furthermore—if desired—can be separated by known methods, especially by distillation and/or chromatography (e. g. fractionation into the individual polyglycerol esters of polycarboxylic acids esterified with oxobutanol, i.e. e.g. separation of the respective monoesters, diesters etc., or else fractionation into fractions with enriched and depleted portions of individuals etc.).

Thus, as previously stated, according to the first aspect, the present invention refers to a method for producing polyglycerol esters of polycarboxylic acids esterified with oxobutanol, especially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol,

wherein
(i) at least one oxobutanol, especially 3-hydroxybutanoate, preferentially 4-oxo-2-butanol, preferably 4-oxo-4-(C₁-C₅-alkoxy)-2-butanol or 4-oxo-4-(hydroxy-C₃-C₅-alkoxy)-2-butanol, of the general formula (I)

CH₃—CH(OH)—CH₂—C(O)OR¹ (I)
wherein, in the general formula (I), the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl,
(ii) at least one polycarboxylic acid (II), especially at least one polycarboxylic acid comprising at least two carboxyl groups, and
(iii) at least one polyglycerol (III)

are reacted and/or caused to react with one another,

so that, as a reaction product (IV), one or more polyglycerol esters of polycarboxylic acids esterified with oxobutanol, especially one or more polyglycerol esters of polycarboxylic acids esterified with 4-oxo-2-butanol, preferentially one or more mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, are obtained.

Especially, the method may be carried out in one step, especially as a one-pot synthesis, or else in several steps, especially in two steps, preferably in several steps, especially in two steps, more preferably in two steps.

According to a preferred embodiment of the present invention, the method may be carried out in several steps, especially in two steps,

wherein
(a) in a first method step (a), at least one oxobutanol, especially 3-hydroxybutanoate, preferentially 4-oxo-2-butanol, preferably 4-oxo-4-(C₁-C₅-alkoxy)-2-butanol or 4-oxo-4-(hydroxy-C₃-C₅-alkoxy)-2-butanol, of the general formula (I)

CH₃—CH(OH)—CH₂—C(O)OR¹ (I)
wherein, in the general formula (I), the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl,
is reacted and/or caused to react with at least one polycarboxylic acid (II), especially with at least one polycarboxylic acid comprising at least two carboxyl groups, especially in an esterification reaction and/or under esterification conditions,
especially so that, as a reaction intermediate product (IV′) of the method step (a), one or more polycarboxylic acid esters of oxobutanol, especially one or more polycarboxylic acid esters of 3-hydroxybutanoate, preferably one or more polycarboxylic acid esters of 4-oxo-2-butanol, preferentially one or more polycarboxylic acid esters of 4-oxo-4-(C₁-C₅-alkoxy)-2-butanol or of 4-oxo-4-(hydroxy-C₃-C₅-alkoxy)-2-butanol, are obtained; and
(b) subsequently, in a second method step (b), the reaction intermediate product (IV′) obtained in method step (a) is reacted and/or caused to react with at least one polyglycerol (III), especially in an esterification reaction and/or under esterification conditions.

According to a particular embodiment, the present invention also refers, in accordance with this aspect of the invention, to a method for producing polyglycerol esters of polycarboxylic acids esterified with oxobutanol, especially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, especially a method as described hereinabove,

wherein:
(a) in a first method step (a), at least one oxobutanol, especially 3-hydroxybutanoate, preferentially 4-oxo-2-butanol, preferably 4-oxo-4-(C₁-C₅-alkoxy)-2-butanol or 4-oxo-4-(hydroxy-C₃-C₅-alkoxy)-2-butanol, of the general formula (I)

CH₃—CH(OH)—CH₂—C(O)OR¹ (I)
wherein, in the general formula (I), the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl,
is reacted and/or caused to react with at least one polycarboxylic acid (II), especially with at least one polycarboxylic acid comprising at least two carboxyl groups, especially in an esterification reaction and/or under esterification conditions,
especially so that, as a reaction intermediate product (IV′) of the method step (a), one or more polycarboxylic acid esters of oxobutanol, especially one or more polycarboxylic acid esters of 3-hydroxybutanoate, preferably one or more polycarboxylic acid esters of 4-oxo-2-butanol, preferentially one or more polycarboxylic acid esters of 4-oxo-4-(C₁-C₅-alkoxy)-2-butanol or of 4-oxo-4-(hydroxy-C₃-C₅-alkoxy)-2-butanol, are obtained; and
(b) subsequently, in a second method step (b), the reaction intermediate product (IV′) obtained in method step (a) is reacted and/or caused to react with at least one polyglycerol (III), especially in an esterification reaction and/or under esterification conditions,
   so that, as a reaction product (IV), one or more polyglycerol esters of polycarboxylic acids esterified with oxobutanol, especially one or more polyglycerol esters of polycarboxylic acids esterified with 4-oxo-2-butanol, especially one or more mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, are obtained.

According to a particular embodiment of the present invention, the compound of the general formula (I) may be used in racemic form or in the form of the (R)-enantiomer. The (R)-configuration refers to the chiral carbon atom in the 3-position of the compound of the general formula (I).

According to the invention, it is preferred if, in the general formula (I), the radical R¹ represents ethyl.

In other words, it is preferred according to the invention that, as compound of the general formula (I), ethyl 3-hydroxybutyrate (3-hydroxybutyric acid ethyl ester or 4-ethoxy-4-oxo-2-butanol) of the formula CH₃—CH(OH)—CH₂—C(O)OC₂H is used.

This enables particularly efficient process control and high yields with minimized or suppressed by-product formation. In addition, the 3-hydroxybutyric acid ethyl ester or 4-ethoxy-4-oxo-2-butanol is also commercially available in large quantities and can be readily obtained, especially on a large scale (e.g. by Claisen condensation of ethyl acetate).

According to a particular embodiment of the present invention, the polycarboxylic acid (II) may be used in the form of the free polycarboxylic acid, in the form of a salt of the polycarboxylic acid, in the form of a polycarboxylic acid ester or in the form of the polycarboxylic acid anhydride, especially in the form of the free polycarboxylic acid or in the form of the polycarboxylic acid anhydride, preferably in the form of the polycarboxylic acid anhydride, more preferably in the form of a cyclic polycarboxylic acid anhydride.

The anhydrides of the polycarboxylic acid are particularly reactive and are especially suitable for esterification reactions. When cyclic anhydrides are used, no cleavage products are formed in the course of an esterification reaction, which may require energy-intensive removal.

According to another particular embodiment of the present invention, the polycarboxylic acid (II) may correspond to the general formula (IIa)

HOOC—X—COOH (IIa)

wherein, in the general formula (IIa), X represents a saturated or unsaturated atoms and optionally mono- or polysubstituted, especially substituted with one or more hydroxyl radicals and/or carboxyl radicals, organic radical comprising 1 to 10, especially 2 to 6 carbon;

especially wherein at least one carboxyl group (COOH-group), preferentially both carboxyl groups (COOH-groups), is/are terminal and/or is/are a primary carboxyl group (COOH-group).

In this context, it is particularly preferred if, in the general formula (IIa), X represents a saturated or unsaturated optionally mono- or polysubstituted, especially substituted with one or more hydroxyl radicals and/or carboxyl radicals, organic radical comprising 2 to 6 carbon atoms and;

especially wherein at least one carboxyl group (COOH-group), especially both carboxyl groups (COOH-groups), is/are terminal and/or is/are a primary carboxyl group (COOOH-group).

Especially, by using a previously defined carboxylic acid with at least one, preferentially two, terminal or primary carboxyl groups, the esterification reaction between the polycarboxylic acid (II) and the oxobutanol of the general formula (I) can proceed particularly effectively and with minimized by-product formation without the need for extreme reaction conditions (e.g. very high temperature, very low pressure etc.). In addition, the active ingredient density (i.e. especially the 3-hydroxybutanoate density) can be influenced by the number of carboxyl groups present.

In the method according to the invention it may be preferred if the polycarboxylic acid (II) is selected from the group of succinic acid, tartaric acid, citric acid, malic acid, adipic acid, fumaric acid and maleic acid and their anhydrides as well as combinations or mixtures thereof, especially selected from the group of succinic acid, tartaric acid, citric acid, malic acid, adipic acid and fumaric acid and their anhydrides as well as their combinations or mixtures, preferably selected from the group of succinic acid and adipic acid and their anhydrides as well as their combinations or mixtures.

The previously mentioned carboxylic acids are particularly suitable for reaction with 3-hydroxybutanoates and are also commercially available.

Especially, it is preferred in the method according to the invention if the polycarboxylic acid (II) is a naturally occurring carboxylic acid or its anhydride or derivative, especially reaction product, especially a carboxylic acid or its anhydride or derivative, especially reaction product, occurring in human and/or animal metabolism.

Especially, it is advantageous in this context if carboxylic acids or anhydrides or derivatives thereof are used which occur in the citrate cycle, result from the citrate cycle or are associated with the citrate cycle. In this context, derivatives can represent, for example, salts or esters which are obtainable by oxidation of a metabolic product (for example, from the citrate cycle). By using carboxylic acids or anhydrides or derivatives thereof, which represent part of the human and/or animal metabolism or reactant or product or intermediate of a human and/or animal metabolism, a further energy source (in addition to the keto body 3-hydroxybutyric acid or 3-hydroxybutanoate) can be provided to the human and/or animal body when using the reaction product according to the invention. The inventive reaction products are particularly suitable for the use in or as medicaments, drugs or food and food products.

Furthermore, it may also be preferred in the method according to the invention if the polycarboxylic acid (II) is an ingredient, especially an additive, approved under food law.

Ingredients or additives approved under food law are permitted for use in food in certain quantities and do not pose any health risks. A list of food additives is maintained throughout the EU, wherein each food additive is given its own label (so-called E-number). For example, the following carboxylic acids are included in the food additive list: succinic acid (E363), tartaric acid (E334), citric acid (E330), malic acid (E296), adipic acid (E355) and fumaric acid (E297). These acids are all part of the citrate cycle or obtainable by oxidation of a metabolic product of the citrate cycle. The citrate cycle is a cycle of biochemical reactions that plays an important role in the metabolism of aerobic cells of living organisms and is mainly used for the oxidative degradation of organic substances for the purpose of energy production and the provision of intermediates for biosynthesis. Thus, the acids formed by degradation when using the reaction product (IV) obtainable from the inventive method can be utilized by the body as another alternative source of energy.

According to a particular embodiment of the method according to the invention, the polyglycerol may correspond to the general formula (IIIa)

HO—CH₂—CH(OH)—CH₂—[O—CH₂—CH(OH)—CH₂]ₚ—OH (IIIa)

wherein, in general formula (IIIa), the variable p represents an integer from 1 to 6, especially from 1 to 4, preferentially 1 or 2, more preferably 1.

According to a further particular embodiment of the inventive method, the polyglycerol (III) may be a diglycerol of formula (IIIb)

HO—CH₂—CH(OH)—CH₂—O—CH₂—CH(OH)—CH₂—OH (IIIb)

Especially, the polyglycerol (III) is not propane-1,2,3-triol (glycerol).

According to a particular embodiment, the present invention refers, in accordance with this aspect of the invention, to a method for producing polyglycerol esters of polycarboxylic acids esterified with oxobutanol, especially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, especially a method as described hereinabove,

wherein:
(a) in a first method step (a), at least one oxobutanol, especially 3-hydroxybutanoate, preferably 4-oxo-2-butanol, preferentially 4-oxo-4-(C₁-C₅-alkoxy)-2-butanol or 4-oxo-4-(hydroxy-C₃-C₅-alkoxy)-2-butanol, of the general formula (I)

CH₃—CH(OH)—CH₂—C(O)OR¹ (I)
wherein, in the general formula (I), the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl,
is reacted and/or caused to react with at least one polycarboxylic acid (II), especially with at least one polycarboxylic acid comprising at least two carboxyl groups, especially in an esterification reaction and/or under esterification conditions,
especially so that, as a reaction intermediate product (IV′) of the method step (a), one or more polycarboxylic acid esters of oxobutanol, especially one or more polycarboxylic acid esters of 3-hydroxybutanoate, preferably one or more polycarboxylic acid esters of 4-oxo-2-butanol, preferentially one or more polycarboxylic acid esters of 4-oxo-4-(C₁-C₅-alkoxy)-2-butanol or of 4-oxo-4-(hydroxy-C₃-C₅-alkoxy)-2-butanol, are obtained; and
(b) subsequently, in a second method step (b), the reaction intermediate product (IV′) obtained in method step (a) is reacted and/or caused to react, especially in an esterification reaction and/or reacted and/or reacted under esterification conditions, with at least one polyglycerol of the general formula (IIIa)

HO—CH₂—CH(OH)—CH₂—[O—CH₂—CH(OH)—CH₂]ₚ—OH (IIIa)

wherein, in the general formula (IIIa), the variable p represents an integer from 1 to 6, especially from 1 to 4, preferentially 1 or 2, more preferably 1,

so that, as a reaction product (IV), one or more polyglycerol esters of polycarboxylic acids esterified with oxobutanol, especially one or more polyglycerol esters of polycarboxylic acids esterified with 4-oxo-2-butanol, preferentially one or more mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, are obtained.

According to a further preferred embodiment, the present invention according to this aspect of the invention also refers to a method for producing polyglycerol esters of polycarboxylic acids esterified with oxobutanol, especially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, especially a method as described hereinabove,

wherein:
(a) in a first method step (a), at least one oxobutanol, especially 3-hydroxybutanoate, preferably 4-oxo-2-butanol, preferentially 4-oxo-4-(C₁-C₅-alkoxy)-2-butanol or 4-oxo-4-(hydroxy-C₃-C₅-alkoxy)-2-butanol, of the general formula (I)

CH₃—CH(OH)—CH₂—C(O)OR¹ (I)
wherein, in the general formula (I), the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl,
is reacted and/or caused to react, especially in an esterification reaction and/or under esterification conditions, with at least one polycarboxylic acid (II) selected from the group of succinic acid, tartaric acid, citric acid, malic acid, adipic acid, fumaric acid and maleic acid and anhydrides thereof as well as combinations or mixtures thereof, especially selected from the group of succinic acid, tartaric acid, citric acid, malic acid, adipic acid and fumaric acid and their anhydrides as well as their combinations or mixtures, preferably selected from the group of succinic acid and adipic acid and anhydrides thereof as well as combinations or mixtures thereof,
especially so that, as a reaction intermediate product (IV′) of the method step (a), one or more polycarboxylic acid esters of oxobutanol, especially one or more polycarboxylic acid esters of 3-hydroxybutanoate, preferably one or more polycarboxylic acid esters of 4-oxo-2-butanol, preferentially one or more polycarboxylic acid esters of 4-oxo-4-(C₁-C₅-alkoxy)-2-butanol or of 4-oxo-4-(hydroxy-C₃-C₅-alkoxy)-2-butanol, are obtained; and
(b) subsequently, in a second method step (b), the reaction intermediate product (IV′) obtained in method step (a) is reacted and/or caused to react, especially in an esterification reaction and/or under esterification conditions, with at least one polyglycerol of the general formula (IIIa)

HO—CH₂—CH(OH)—CH₂—[O—CH₂—CH(OH)—CH₂]ₚ—OH (IIIa)
wherein, in the general formula (IIIa), the variable p represents an integer from 1 to 6, especially from 1 to 4, preferentially 1 or 2, more preferably 1,
so that, as a reaction product (IV), one or more polyglycerol esters of polycarboxylic acids esterified with oxobutanol, especially one or more polyglycerol esters of polycarboxylic acids esterified with 4-oxo-2-butanol, preferentially one or more mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, are obtained.

According to a particular embodiment of the present invention, the method may be carried out in the absence of solvents and/or without any solvent

According to a further particular embodiment of the present invention, especially the first method step (a) and/or the second method step (b), especially the first method step (a) and the second method step (b), can be carried out in the absence of solvents and/or without any solvent

I.e. the method or the first method step (a) and/or the second method step (b) is/are thus carried out as a reaction in bulk or as a reaction in substance or as a so-called bulk reaction. This has the advantage that the reaction products obtained are not contaminated with solvent and no solvent has to be removed and disposed of or recycled in a costly and energy-intensive manner after the method or reaction has been carried out Surprisingly, the method or reaction nevertheless proceeds with high conversions and yields and at least essentially without significant by-product formation.

According to a particular embodiment of the present invention, the method may be carried out in a single step.

According to a particular embodiment, it is possible that the method can be carried out in the absence of a catalyst and/or without any catalyst, or else the method is carried out in the presence of a catalyst, especially an enzyme and/or a metal-containing and/or metal-based, acidic or basic catalyst (especially wherein the catalyst is recycled after reaction).

As previously stated, according to a particular embodiment of the inventive production method, the method may be carried out in the absence of a catalyst and/or without any catalyst.

Provided that the reaction is carried out in the absence of a catalyst and/or without any catalyst, it is preferred if the reaction is carried out at temperatures in the range of from 20° C. to 160° C., especially in the range of from 50° C. to 150° C., preferentially in the range of from 70° C. to 140° C., more preferably in the range of from 80° C. to 135° C., even more preferably in the range of from 100° C. to 130° C.

In the case of conversion in the absence of a catalyst, the applied pressure range can vary within wide ranges. Especially, the reaction may be carried out in the absence of a catalyst and/or without any catalyst at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 1 bar.

When reacting in the absence of a catalyst, it is preferred if the reaction is carried out in the presence of an inert gas, especially in the presence of helium, argon or nitrogen, preferably in the presence of nitrogen. Especially, undesirable side reactions, especially due to oxidation or hydrolysis, can thus be prevented.

Alternatively to this particular embodiment, however, it is also possible to carry out the reaction in the presence of an enzyme as a catalyst

Especially, the enzyme may be selected from synthetases (ligases), catalases, esterases, lipases and combinations thereof. According to the invention, synthetases (synonymously ligases) are especially enzymes from the class of ligases; ligases are enzymes which catalyze the linking of two or more molecules by a covalent bond. Catalases in the sense of the present invention are especially enzymes which are capable of converting hydrogen peroxide to oxygen and water. The term esterases refers in particular to enzymes which are capable of hydrolytically splitting esters into alcohol and acid (saponification); these are thus especially hydrolases, wherein fat splitting esterases are also called lipases. Lipases in the sense of the present invention are especially enzymes which are capable of splitting free fatty acids from lipids such as glycerides (lipolysis).

In this context, the enzyme used as catalyst may be derived from Candida antarctica, Mucor miehei (Rhizomucor miehei), Thermomyces lanuginosus, Candida rugosa, Aspergillus oryzae, Pseudomonas cepacia, Pseudomonas fluorescens, Rhizopus delemar and Pseudomonas sp. and combinations thereof, preferentially of Candida antarctica, Mucor miehei (Rhizomucor miehei) and Thermomyces lanuginosus.

According to a particular embodiment, the enzyme may be used in immobilized form, especially immobilized on a carrier, preferentially on a polymeric carrier, preferably on a polymeric organic carrier, more preferably with hydrophobic properties, even more preferably on a poly(meth)acrylic resin-based carrier.

In the context of the present invention, it is preferred that in the case that an enzyme is used as a catalyst, the enzyme is recycled after the reaction.

Insofar as the reaction in the production method according to the invention is carried out in the presence of an enzyme as a catalyst, it is preferred if the reaction is carried out at temperatures in the range of from 10° C. to 80° C., especially in the range of from 20° C. to 80° C., preferentially in the range of from 25° C. to 75° C., more preferably in the range of from 45° C. to 75° C., even more preferably in the range of from 50° C. to 70° C.

In case of using an enzyme as a catalyst, the amount of the enzyme used can vary within wide ranges. Especially, the enzyme may be used in amounts, based on the total amount of starting compounds (I) and (II), in the range of from 0.001% by weight to 20% by weight, especially in the range of from 0.01% by weight to 15% by weight, preferentially in the range of from 0.1% by weight to 15% by weight, preferably in the range of from 0.5% by weight to 10% by weight Nevertheless, it may be necessary to deviate from the above-mentioned amounts in individual cases or for specific applications without leaving the scope of the present invention.

When, according to a particular embodiment of the present invention, the reaction is carried out in the presence of an enzyme as a catalyst, the applied pressure range may also vary within wide ranges. Typically, the reaction in the presence of an enzyme as a catalyst can be carried out at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 0.5 bar.

According to the particular embodiment of the present invention, according to which the reaction is carried out in the presence of an enzyme as a catalyst, it is preferred if the reaction is carried out in the presence of an inert gas, especially in the presence of helium, argon or nitrogen, preferably in the presence of nitrogen. As previously stated in connection with the reaction in the absence of a catalyst, the reaction in the presence of an inert gas can prevent undesirable side reactions, especially those due to oxidation or hydrolysis.

According to another alternative embodiment of the present invention, the reaction may be carried out in the presence of a metal-containing and/or metal-based acidic or basic catalyst.

According to this alternative embodiment of the present invention, according to which the reaction is carried out in the presence of a metal-containing and/or metal-based, acidic or basic catalyst, the catalyst may especially be selected from (i) basic catalysts, especially alkali or alkaline earth hydroxides and alkali or alkaline earth alcoholates, such as NaOH, KOH, LiOH, Ca(OH)₂, NaOMe, KOMe and Na(OBu-tert.), (ii) acidic catalysts, especially mineral acids, and organic acids, such as sulfuric acid, hydrochloric acid, phosphoric acid, nitric acid, sulfonic acids, methane sulfonic acid, para-toluene sulfonic acid and carboxylic acids, (iii) Lewis acids, especially Lewis acids based on titanium, tin, zinc and aluminum compounds, such as titanium tetrabutylate, tin acids, zinc acetate, aluminum trichloride and aluminum tri-isopropyl, and (iv) heterogeneous catalysts, especially based on mineral silicates, germanates, carbonates and aluminum oxides, such as zeolites, montmorillonites, mordenites, hydrotalcites and aluminas, and combinations thereof.

In this embodiment, a Lewis acid based on titanium, tin, zinc and aluminum compounds, such as titanium tetrabutylate, tin acids, zinc acetate, aluminum trichloride and aluminum tri-isopropyl, may be used as a catalyst.

Especially, it is also preferred in this embodiment that the metal-containing and/or metal-based acidic or basic catalyst is recycled after the reaction.

Also according to the particular embodiment of the present invention, according to which the reaction is carried out in the presence of a metal-containing and/or metal-based, acidic or basic catalyst, the temperatures can be varied within wide ranges. Especially, the reaction may be carried out in the presence of a metal-containing and/or metal-based, acidic or basic catalyst at temperatures in the range of from 20° C. to 160° C., especially in the range of from 50° C. to 150° C., preferentially in the range of from 70° C. to 140° C., more preferably in the range of from 80° C. to 135° C., even more preferably in the range of from 100° C. to 130° C.

Furthermore, also in this embodiment, the catalyst (i.e. the metal-containing and/or metal-based, acidic or basic catalyst) can be varied within wide quantity ranges: Thus, the catalyst can be used in amounts, based on the total amount of starting compounds (I), (II) and (III), in the range of from 0.01% by weight to 30% by weight, especially in the range of from 0.05% by weight to 15% by weight, preferentially in the range of from 0.1% by weight to 15% by weight, preferably in the range of from 0.2% by weight to 10% by weight Nevertheless, it is possible to deviate from the above-mentioned amounts in individual cases or for specific applications without leaving the scope of the present invention.

Moreover, according to this particular embodiment of the present invention, according to which the reaction is carried out in the presence of a metal-containing and/or metal-based, acidic or basic catalyst, the pressure range may equally vary within wide ranges: Especially, the reaction in the presence of a metal-containing and/or metal-based, acidic or basic catalyst can be carried out at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 1 bar.

Furthermore, also according to this particular embodiment of the present invention, according to which the reaction is carried out in the presence of a metal-containing and/or metal-based, acidic or basic catalyst, it is preferred if the reaction is carried out in the presence of an inert gas, especially in the presence of helium, argon or nitrogen, preferably in the presence of nitrogen. As previously stated, the reaction in the presence of an inert gas prevents undesirable side reactions, especially due to oxidation or hydrolysis.

According to a particular embodiment, in the multistage, especially two-stage procedure, the first method step (a) may be carried out in the absence of a catalyst and/or without any catalyst, or else in the presence of a catalyst, especially an enzyme and/or a metal-containing and/or metal-based, acidic or basic catalyst, especially wherein the catalyst is recycled after the reaction.

As previously stated, according to a particular embodiment of the production method according to the invention, in the multistage, especially two-stage procedure, the first method step (a) may also be carried out in the absence of a catalyst and/or without any catalyst.

Insofar as the first method step (a) is carried out in the absence of a catalyst and/or without any catalyst, it is preferred if the first method step (a) is carried out in the absence of a catalyst and/or without any catalyst at temperatures in the range of from 20° C. to 160° C., especially in the range of from 50° C. to 150° C., preferentially in the range of from 70° C. to 140° C., more preferably in the range of from 80° C. to 135° C., even more preferably in the range of from 100° C. to 130° C.

In the case of carrying out the first method step (a) in the absence of a catalyst, the applied pressure range can vary within wide ranges. Especially, the first method step (a) may be carried out in the absence of a catalyst and/or without any catalyst at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 1 bar.

When carrying out the first method step (a) in the absence of a catalyst, it is preferred if the first method step (a) is carried out in the absence of a catalyst and/or without any catalyst in the presence of an inert gas, especially in the presence of helium, argon or nitrogen, preferably in the presence of nitrogen.

As stated above, it is also possible as an alternative to this particular embodiment, to carry out the first method step (a) in the multistage, especially two-stage procedure in the presence of an enzyme as a catalyst.

Especially, the enzyme may be selected from synthetases (ligases), catalases, esterases, lipases and combinations thereof. According to the invention, synthetases (synonymously ligases) are especially enzymes from the class of ligases; ligases are enzymes which catalyze the linking of two or more molecules by a covalent bond. Catalases in the sense of the present invention are especially enzymes which are capable of converting hydrogen peroxide to oxygen and water. The term esterases refers in particular to enzymes which are capable of hydrolytically splitting esters into alcohol and acid (saponification); these are thus especially hydrolases, wherein fat splitting esterases are also called lipases. Lipases in the sense of the present invention are especially enzymes which are capable of splitting free fatty acids from lipids such as glycerides (lipolysis).

In this context, the enzyme used as catalyst can be derived from Candida antarctica, Mucor miehei (Rhizomucor miehei), Thermomyces lanuginosus, Candida rugosa, Aspergillus oryzae, Pseudomonas cepacia, Pseudomonas fluorescens, Rhizopus delemar and Pseudomonas sp. and combinations thereof, preferentially from Candida antarctica, Mucor miehei (Rhizomucor miehei) and Thermomyces lanuginosuss.

According to a particular embodiment, the enzyme can be used in immobilized form, immobilized on a carrier, preferentially on a polymeric carrier, preferably on a polymeric organic carrier, more preferably with hydrophobic properties, even more preferably on a poly(meth)acrylic resin-based carrier.

In the context of the present invention, it is preferred that in case an enzyme is used as a catalyst, the enzyme is recycled after the reaction.

Insofar as, in the multistage, especially two-stage procedure, the first method step (a) in the inventive production method is carried out in the presence of an enzyme as a catalyst, it is preferred if the first method step (a) is carried out at temperatures in the range of from 10° C. to 80° C., especially in the range of from 20° C. to 80° C., preferentially in the range of from 25° C. to 75° C., more preferably in the range of from 45° C. to 75° C., even more preferably in the range of from 50° C. to 70° C.

In the case of using an enzyme as a catalyst, the amount of the enzyme used can vary within wide ranges. Especially, the enzyme can be used in amounts, based on the total amount of starting compounds (I) and (II), in the range of from 0.001% by weight to 20% by weight, especially in the range of from 0.01% by weight to 15% by weight, preferentially in the range of from 0.1% by weight to 15% by weight, preferably in the range of from 0.5% by weight to 10% by weight Nevertheless, it may be necessary to deviate from the above-mentioned amounts in individual cases or for specific applications without leaving the scope of the present invention.

If, according to this particular embodiment of the present invention, the first method step (a) is carried out in the presence of an enzyme as a catalyst, the applied pressure range can also vary within wide ranges. Typically, the first method step (a) can be carried out in the presence of an enzyme as a catalyst at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 0.5 bar.

According to this particular embodiment of the present invention, according to which, in the multistage, especially two-stage procedure, the first method step (a) is carried out in the presence of an enzyme as a catalyst, it is preferred if the first method step (a) is carried out in the presence of an inert gas, especially in the presence of helium, argon or nitrogen, preferably in the presence of nitrogen. As previously stated in connection with the reaction in the absence of a catalyst, undesirable side reactions, especially due to oxidation or hydrolysis, can be prevented by the reaction in the presence of an inert gas.

According to a further alternative embodiment of the present invention, in the multistage, especially two-stage procedure, the first method step (a) can be carried out in the presence of a metal-containing and/or metal-based, acidic or basic catalyst.

According to this alternative embodiment of the present invention, according to which the reaction is carried out in the presence of a metal-containing and/or metal-based, acidic or basic catalyst, the catalyst can especially be selected from (i) basic catalysts, especially alkali or alkaline earth hydroxides and alkali or alkaline earth alcoholates, such as NaOH, KOH, LiOH, Ca(OH)₂, NaOMe, KOMe and Na(OBu-tert.), (ii) acidic catalysts, especially mineral acids, and organic acids, such as sulfuric acid, hydrochloric acid, phosphoric acid, nitric acid, sulfonic acids, methane sulfonic acid, para-toluene sulfonic acid and carboxylic acids, (iii) Lewis acids, especially Lewis acids based on titanium, tin, zinc and aluminum compounds, such as titanium tetrabutylate, tin acids, zinc acetate, aluminum trichloride and aluminum tri-isopropyl, and (iv) heterogeneous catalysts, especially based on mineral silicates, germanates, carbonates and aluminum oxides, such as zeolites, montmorillonites, mordenites, hydrotalcites and aluminas, and combinations thereof.

In this embodiment, a Lewis acid based on titanium, tin, zinc and aluminum compounds, such as titanium tetrabutylate, tin acids, zinc acetate, aluminum trichloride and aluminum tri-isopropyl, may be used as a catalyst

Especially, it is also preferred in this embodiment if the metal-containing and/or metal-based acidic or basic catalyst is recycled after the reaction.

Also according to the particular embodiment of the present invention, according to which in the multistage, especially two-stage procedure the first method step (a) is carried out in the presence of a metal-containing and/or metal-based, acidic or basic catalyst, the temperatures can be varied within wide ranges. Especially, the first method step (a) can be carried out in the presence of a metal-containing and/or metal-based, acidic or basic catalyst at temperatures in the range of from 20° C. to 160° C., especially in the range of from 50° C. to 150° C., preferentially in the range of from 70° C. to 140° C., more preferably in the range of from 80° C. to 135° C., even more preferably in the range of from 100° C. to 130° C.

Furthermore, also in this embodiment, the catalyst (i.e. the metal-containing and/or metal-based, acidic or basic catalyst) can be varied within wide quantity ranges: Thus, the catalyst can be used in amounts, based on the total amount of starting compounds (I) and (II), in the range of from 0.01% by weight to 30% by weight, especially in the range of from 0.05% by weight to 15% by weight, preferentially in the range of from 0.1% by weight to 15% by weight, preferably in the range of from 0.2% by weight to 10% by weight Nevertheless, it is possible to deviate from the above-mentioned amounts in individual cases or for specific applications without leaving the scope of the present invention.

Furthermore, according to this particular embodiment of the present invention, according to which, in the multistage, especially two-stage procedure, the first method step (a) is carried out in the presence of a metal-containing and/or metal-based, acidic or basic catalyst, the pressure range can equally vary within wide ranges: Especially, the first method step (a) can be carried out in the presence of a metal-containing and/or metal-based, acidic or basic catalyst at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 1 bar.

Furthermore, also according to this particular embodiment of the present invention, according to which in the multistage, especially two-step procedure the first method step (a) is carried out in the presence of a metal-containing and/or metal-based, acidic or basic catalyst, it is preferred if the reaction is carried out in the presence of an inert gas, especially in the presence of helium, argon or nitrogen, preferably in the presence of nitrogen. As previously stated, the reaction in the presence of an inert gas prevents undesirable side reactions, especially due to oxidation or hydrolysis.

Furthermore, according to another particular embodiment of the present invention, in the multistage, especially two-step procedure, the second method step (b) may be carried out in the absence of a catalyst and/or without any catalyst, or else may be carried out in the presence of a catalyst, especially an enzyme and/or a metal-containing and/or metal-based, acidic or basic catalyst (especially wherein the catalyst is recycled after the reaction).

As previously stated, according to a particular embodiment of the production method according to the invention, the second method step (b) in the multistage, especially two-stage procedure can also be carried out in the absence of a catalyst and/or without any catalyst.

Insofar as in the multistage, especially two-stage procedure the second method step (b) is carried out in the absence of a catalyst and/or without any catalyst, it is preferred if the second method step (b) is carried out in the absence of a catalyst and/or without any catalyst at temperatures in the range of from 20° C. to 160° C., especially in the range of from 50° C. to 150° C., preferentially in the range of from 70° C. to 140° C., more preferably in the range of from 80° C. to 135° C., even more preferably in the range of from 100° C. to 130° C.

In case of carrying out the second method step (b) in the absence of a catalyst, the applied pressure range can vary within wide ranges. Especially, the second method step (b) can be carried out in the absence of a catalyst and/or without any catalyst at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 1 bar.

When carrying out the second method step (b) in the absence of a catalyst, it is preferred if the second method step (b) is carried out in the presence of an inert gas, especially in the presence of helium, argon or nitrogen, preferably in the presence of nitrogen. Especially, undesirable side reactions, especially due to oxidation or hydrolysis, can thus be prevented.

Alternatively to this particular embodiment, however, it is also possible to carry out the second method step (b) in the multistage, especially two-step procedure in the presence of an enzyme as a catalyst Especially, the enzyme may be selected from synthetases (ligases), catalases, esterases, lipases and combinations thereof. According to the invention, synthetases (synonymously ligases) are especially enzymes from the class of ligases; ligases are enzymes which catalyze the linking of two or more molecules by a covalent bond. Catalases in the sense of the present invention are especially enzymes which are capable of converting hydrogen peroxide to oxygen and water. The term esterases refers in particular to enzymes which are capable of hydrolytically splitting esters into alcohol and acid (saponification); these are thus especially hydrolases, wherein fat splitting esterases are also called lipases. Lipases in the sense of the present invention are especially enzymes which are capable of splitting free fatty acids from lipids such as glycerides (lipolysis).

In this context, the enzyme used as catalyst can be derived especially from Candida antarctica, Mucor miehei (Rhizomucor miehei), Thermomyces lanuginosus, Candida rugosa, Aspergillus oryzae, Pseudomonas cepacia, Pseudomonas fluorescens, Rhizopus delemar and Pseudomonas sp. and combinations thereof, preferentially of Candida antarctica, Mucor miehei (Rhizomucor miehei) and Thermomyces lanuginosus.

According to a particular embodiment, the enzyme may be used in immobilized form, especially immobilized on a carrier, preferentially on a polymeric carrier, preferably on a polymeric organic carrier, more preferably with hydrophobic properties, even more preferably on a poly(meth)acrylic resin-based carrier.

In the context of the present invention, it is preferred that in case an enzyme is used as a catalyst, the enzyme is recycled after the reaction.

Insofar as in the multistage, especially two-stage procedure the second method step (b) in the production method according to the invention is carried out in the presence of an enzyme as a catalyst, it is preferred if the second method step (b) is carried out in the presence of an enzyme as a catalyst at temperatures in the range of from 10° C. to 80° C., especially in the range of from 20° C. to 80° C., preferentially in the range of from 25° C. to 75° C., more preferably in the range of from 45° C. to 75° C., even more preferably in the range of from 50° C. to 70° C.

In the case of using an enzyme as a catalyst, the amount of enzyme used can vary within wide ranges. Especially, the enzyme can be used in amounts, based on the total amount of the starting compounds (IV) and (III), in the range of from 0.001% by weight to 20% by weight, especially in the range of from 0.01% by weight to 15% by weight, preferentially in the range of from 0.1% by weight to 15% by weight, preferably in the range of from 0.5% by weight to 10% by weight Nevertheless, it may be necessary to deviate from the above-mentioned amounts in individual cases or for specific applications without leaving the scope of the present invention.

If, according to a particular embodiment of the present invention, the second method step (b) is carried out in the presence of an enzyme as a catalyst in the multistage, especially two-stage procedure, the pressure range applied can also vary within wide ranges. Typically, the second method step (b) can be carried out in the presence of an enzyme as a catalyst at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 0.5 bar.

According to the particular embodiment of the present invention, according to which the second method step (b) is carried out in the presence of an enzyme as a catalyst, it is preferred if the second method step (b) is carried out in the presence of an enzyme in the presence of an inert gas, especially in the presence of helium, argon or nitrogen, preferably in the presence of nitrogen. As previously stated in connection with the reaction in the absence of a catalyst, undesirable side reactions, especially due to oxidation or hydrolysis, can be prevented by the reaction in the presence of an inert gas.

According to a further alternative embodiment of the present invention, in the multistage, especially two-step procedure, the second method step (b) can be carried out in the presence of a metal-containing and/or metal-based, acidic or basic catalyst.

According to this alternative embodiment of the present invention, according to which the reaction is carried out in the presence of a metal-containing and/or metal-based, acidic or basic catalyst, the catalyst may especially be selected from (i) basic catalysts, especially alkali or alkaline earth hydroxides and alkali or alkaline earth alcoholates, such as NaOH, KOH, LiH, Ca(OH)₂, NaOMe, KOMe and Na(OBu-tert.), (ii) acidic catalysts, especially mineral acids, and organic acids, such as sulfuric acid, hydrochloric acid, phosphoric acid, nitric acid, sulfonic acids, methane sulfonic acid, para-toluene sulfonic acid and carboxylic acids, (iii) Lewis acids, especially Lewis acids based on titanium, tin, zinc and aluminum compounds, such as titanium tetrabutylate, tin acids, zinc acetate, aluminum trichloride and aluminum tri-isopropyl, and (iv) heterogeneous catalysts, especially based on mineral silicates, germanates, carbonates and aluminum oxides, such as zeolites, montmorillonites, mordenites, hydrotalcites and aluminas, and combinations thereof.

In this embodiment, a Lewis acid based on titanium, tin, zinc and aluminum compounds, such as titanium tetrabutylate, tin acids, zinc acetate, aluminum trichloride and aluminum tri-isopropyl, may be used as a catalyst.

Especially, it is also preferred in this embodiment if the metal-containing and/or metal-based acidic or basic catalyst is recycled after the reaction.

Also according to the particular embodiment of the present invention, according to which in the multistage, especially two-stage procedure the second method step (b) is carried out in the presence of a metal-containing and/or metal-based, acidic or basic catalyst, the temperatures can be varied within wide ranges. Especially, the second method step (b) can be carried out in the presence of a metal-containing and/or metal-based, acidic or basic catalyst at temperatures in the range of from 20° C. to 160° C., especially in the range of from 50° C. to 150° C., preferentially in the range of from 70° C. to 140° C., more preferably in the range of from 80° C. to 135° C., even more preferably in the range of from 100° C. to 130° C.

Furthermore, also in this embodiment, the catalyst (i.e. the metal-containing and/or metal-based, acidic or basic catalyst) can be varied within wide quantity ranges: Thus, the catalyst can be used in amounts, based on the total amount of starting compounds (IV′) and (III), in the range of from 0.01% by weight to 30% by weight, especially in the range of from 0.05% by weight to 15% by weight, preferentially in the range of from 0.1% by weight to 15% by weight, preferably in the range of from 0.2% by weight to 10% by weight Nevertheless, it is possible to deviate from the above-mentioned amounts in individual cases or for specific applications without leaving the scope of the present invention.

Furthermore, according to this particular embodiment of the present invention, according to which, in the multistage, especially two-stage procedure, the second method step (b) is carried out in the presence of a metal-containing and/or metal-based, acidic or basic catalyst, the pressure range can equally vary within wide ranges: Especially, the second method step (b) can be carried out in the presence of a metal-containing and/or metal-based, acidic or basic catalyst at a pressure in the range of from 0.0001 bar to 10 bar, especially in the range of from 0.001 bar to 5 bar, preferentially in the range of from 0.01 bar to 2 bar, more preferably in the range of from 0.05 bar to 1 bar, even more preferably at about 1 bar.

Furthermore, also according to this particular embodiment of the present invention, according to which in the multistage, especially two-stage procedure the second method step (b) is carried out in the presence of a metal-containing and/or metal-based, acidic or basic catalyst, it is preferred if the second method step (b) is carried out in the presence of an inert gas, especially in the presence of helium, argon or nitrogen, preferably in the presence of nitrogen. As previously stated, the reaction in the presence of an inert gas prevents undesirable side reactions, especially due to oxidation or hydrolysis.

As far as the amounts of reactants or starting compounds as a whole are concerned, these can be varied within wide ranges.

Taking into account process economy and optimization of the process sequence, especially with regard to minimization of by-products, it is advantageous if, in the first method step (a), the starting compounds (I) and (II) are used in an (I)/(II) molar ratio of about 1:1.

Equally taking into account process economy and optimization of the process sequence, especially with regard to minimization of by-products, it is advantageous if, in the second method step (b), the starting compound (III) and the reaction intermediate product (IV′) are used in a (III)/(IV′) molar ratio in the range of from (1/n):1 to 1:1, wherein n denotes the number of hydroxyl groups of the polyglycerol (III) and is preferentially in the range of from 4 to 9.

Typically, in the method according to the invention, when the polycarboxylic acid (II) is used in the form of the free acid, water is formed simultaneously during the reaction. Especially, it is preferred if the water is withdrawn from the reaction, especially continuously withdrawn, especially by means of preferentially continuous, especially distillative or adsorptive removal.

Usually, when the polycarboxylic acid (II) is used in the form of the anhydride, the corresponding free polycarboxylic acid (II) and water are formed in the inventive method. Especially, it is preferred if the resulting free polycarboxylic acid (II) is further reacted or, after the reaction has taken place, is removed and optionally recycled, especially depending on the amounts and/or proportions of the starting compounds (I), (II) and (III) used, and/or especially wherein the water is withdrawn from the reaction, especially continuously withdrawn, especially by means of preferentially continuous, especially distillative or adsorptive removal.

However, when using internal or cyclic anhydrides (such as succinic acid anhydride or maleic acid anhydride), the ring is opened and no cleavage product is formed, so that the reaction product has a terminal free acid. This method is illustrated below using the example of the reaction of maleic anhydride with ethyl 3-hydroxybutyric acid ester as esterification alcohol:

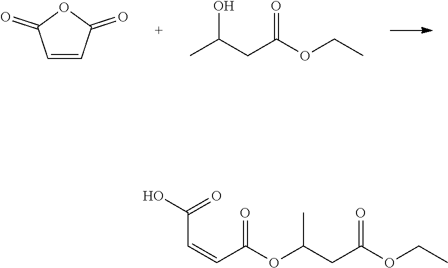

Alternatively, however, the polycarboxylic acid (II) can also be used in the form of the ester. Usually, when the polycarboxylic acid (II) is used in the form of the ester, the corresponding ester alcohol and water are formed in the inventive method. Especially, it is preferred if the resulting ester alcohol and water are withdrawn from the reaction, especially continuously withdrawn, especially by means of preferentially continuous, especially distillative or adsorptive removal.

Continuous removal of by-products (i.e. water and/or ester alcohol) shifts the chemical equilibrium, increasing yield or product formation and minimizing by-product formation.

Within the scope of the production method according to the invention, the composition of the reaction product, especially the presence of the various polyglycerol esters of polycarboxylic acids esterified with oxobutanol, and their proportion in the case of a mixture, may be controlled and/or regulated by means of the reaction conditions, especially by selecting the reaction temperature (conversion temperature) and/or selecting the reaction pressure (conversion pressure) and/or absence of or by providing a catalyst and selecting such catalyst with respect to the type and/or amount and/or by selecting the amounts of the starting compounds (reactants) and/or by providing the removal of the optionally formed by-products.

Thus, it is possible to tailor the composition of the product or product mixture depending on the application; especially, for example, the density of keto bodies (i.e. oxobutanols or 3-hydroxybutanoates) per molecule can be adjusted in a targeted manner.

Following the reaction, the reaction product obtained can be subjected to further conventional or known purification or work-up steps.

In this context, the reaction product obtained can, for example, be fractionated after the reaction has been performed, especially fractionated by distillation.

Also, unreacted starting compounds, especially unreacted starting compounds (I) and/or (II) and/or (III), can be separated from the reaction product and subsequently recycled.

According to a particular embodiment of the production method according to the invention, it is possible to proceed especially in such a way that hydroxyl groups and/or carboxyl groups still present in the reaction product after the reaction has been performed are at least partially, preferentially completely, functionalized, especially esterified.

In other words, the reaction can be followed by a partial, especially complete functionalization, especially esterification, of hydroxyl groups and/or carboxyl groups still present

In this particular embodiment of the method according to the invention, the functionalization, especially the esterification of hydroxyl groups and/or carboxyl groups still present, can be carried out by reaction with a carboxylic acid anhydride of, for example, C₂-C₃₀-carboxylic acids or with C₂-C₃₀-fatty acids or with C₂-C₃₀-fatty alcohols. These can be linear or branched, saturated or mono- or polyunsaturated C₂-C₃₀-carboxylic acid anhydrides or C₂-C₃₀-fatty acids or C₂-C₃₀-fatty alcohols. In this context, hydroxyl groups still present can be reacted especially with carboxylic acid anhydrides or fatty acids, and carboxyl groups still present can be reacted especially with fatty alcohols.

If the method according to the invention is carried out in two steps in accordance with a particular embodiment, as a reaction intermediate product (IV) of method step (a), one or more polycarboxylic acid esters of oxobutanol, especially one or more polycarboxylic acid esters of 3-hydroxybutanoate, preferably one or more polycarboxylic acid esters of 4-oxo-2-butanol, preferentially one or more polycarboxylic acid esters of 4-oxo-4-(C₁-C₅-alkoxy)-2-butanol or of 4-oxo-4-(hydroxy-C₃-C₅-alkoxy)-2-butanol, may be obtained and/or may be obtainable.

Especially, according to the particular embodiment of the method according to the invention, according to which the method is carried out in two steps, as a reaction intermediate product (IV′) of method step (a), one or more polycarboxylic acid esters of oxobutanol, especially one or more polycarboxylic acid esters of 3-hydroxybutanoate, preferably one or more polycarboxylic acid esters of 4-oxo-2-butanol, preferentially one or more polycarboxylic acid esters of 4-oxo-4-(C₁-C₅-alkoxy)-2-butanol or of 4-oxo-4-(hydroxy-C₃-C₅-alkoxy)-2-butanol, of the general formula (IVa′)

R¹—O—C(O)—CH₂—CH(CH₃)—O—(O)C—X—COOH (IVa′)

may be obtained and/or may be obtainable, wherein, in the general formula (IVa′),
R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl;
X represents a saturated or unsaturated and optionally mono- or polysubstituted, especially substituted with one or more hydroxyl radicals and/or radicals —O—C(O)—CH₂—CH(OH)—CH, and/or carboxyl radicals and/or radicals —C(O)—O—CH(CH₃)—CH₂—C(O)OR¹ substituted with R¹ as defined hereinabove, organic radical comprising 1 to 10, preferentially 2 to 6 carbon atoms.

Especially, it is preferred if the radical —COOH is terminal and/or primary radical.

Furthermore, it may be preferred if, according to the particular embodiment of the method according to the invention, according to which the method is carried out in two steps, as a reaction intermediate product (IV′) of method step (a), one or more polycarboxylic acid esters of oxobutanol, especially one or more polycarboxylic acid esters of 3-hydroxybutanoate, preferably one or more polycarboxylic acid esters of 4-oxo-2-butanol, preferentially one or more polycarboxylic acid esters of 4-oxo-4-(C₁-C₅-alkoxy)-2-butanol or of 4-oxo-4-(hydroxy-C₃-C₅-alkoxy)-2-butanol, of the general formula (IVa′)

R¹—O—C(O)—CH₂—CH(CH₃)—O—(O)C—X—COOH (IVa′)

may be obtained and/or may be obtainable, wherein, in the general formula (IVa′),
R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl;
X represents a saturated or unsaturated and optionally mono- or polysubstituted, especially substituted with one or more hydroxyl radicals and/or radicals —O—C(O)—CH₂—CH(OH)—CH₃ and/or carboxyl radicals and/or radicals —C(O)—O—CH(CH₃)—CH₂—C(O)OR¹ substituted with R¹ as defined hereinabove, organic radical comprising 2 to 6 carbon atoms;

especially wherein the radical —COOH is terminal and/or primary radical.

Furthermore, it may also be preferred if, according to the particular embodiment of the method according to the invention, according to which the method is carried out in two steps, as reaction intermediate product (IV′) of method step (a), one or more polycarboxylic acid esters of oxobutanol, especially one or more polycarboxylic acid esters of 3-hydroxybutanoate, preferably one or more polycarboxylic acid esters of 4-oxo-2-butanol, preferentially one or more polycarboxylic acid esters of 4-oxo-4-(C₁-C₅-alkoxy)-2-butanol or of 4-oxo-4-(hydroxy-C₃-C₅-alkoxy)-2-butanol, of the general formula (IVa′)

R¹—O—C(O)—CH₂—CH(CH₃)—O—(O)C—X—COOH (IVa′)

is/are obtained and/or is/are obtainable, wherein, in the general formula (IVa′),
R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl;
X represents a saturated or unsaturated and optionally mono- or polysubstituted, especially substituted with one or more hydroxyl radicals and/or carboxyl radicals, organic radical comprising 2 to 6 carbon atoms;

especially wherein the radical —COOH is terminal and/or is a primary radical.

According to the particular embodiment of the method according to the invention, according to which the method is carried out in two steps, as a reaction intermediate product (IIV′) of method step (a), one or more polycarboxylic acid esters of oxobutanol, especially one or more polycarboxylic acid esters of 3-hydroxybutanoate, preferably one or more polycarboxylic acid esters of 4-oxo-2-butanol, preferentially one or more polycarboxylic acid esters of 4-oxo-4-(C₁-C₅-alkoxy)-2-butanol or of 4-oxo-4-(hydroxy-C₃-C₅-alkoxy)-2-butanol, of the general formula (IVb′)

CH₃—CH(OR²)—CH₂—C(O)OR¹ (IVb′)

may be obtained and/or may be obtainable, wherein, in the general formula (IIIb′),
the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and
the radical R² is derived from a polycarboxylic acid selected from the group of succinic acid, tartaric acid, citric acid, malic acid, adipic acid, fumaric acid and maleic acid as well as combinations or mixtures thereof, especially selected from the group of succinic acid, tartaric acid, citric acid, malic acid, adipic acid and fumaric acid as well as combinations or mixtures thereof, preferably selected from the group of succinic acid and adipic acid and anhydrides thereof as well as combinations or mixtures thereof;

especially wherein, in case of citric acid, a further carboxyl group is esterified with a radical —CH(CH₃)—CH₂—C(O)OR¹ with R¹ as defined hereinabove.

In this context, “derived from” means that the radical R² is formed from the carboxylic acids mentioned; especially, the hydrogen of the carboxyl is esterified by esterification; i.e. in each case the carboxylate radical of the corresponding acid is present as the radical R² (i.e. the radical R² is a succinate radical in the case of succinic acid, a tartrate radical in the case of tartaric acid, a citrate radical in the case of citric acid, a malate radical in the case of malic acid, an adipate radical in the case of adipic acid, a fumarate radical in the case of fumaric acid and a maleate radical in the case of maleic acid).

Furthermore, according to the particular embodiment of the method according to the invention, according to which the method is carried out in two steps, as a reaction intermediate product (IV′) of method step (a), one or more polycarboxylic acid esters of oxobutanol, especially one or more polycarboxylic acid esters of 3-hydroxybutanoate, preferably one or more polycarboxylic acid esters of 4-oxo-2-butanol, preferentially one or more polycarboxylic acid esters of 4-oxo-4-(C₁-C₅-alkoxy)-2-butanol or of 4-oxo-4-(hydroxy-C₃-C₅-alkoxy)-2-butanol, of the general formula (IVb′)

CH₃—CH(OR²)—CH²—C(O)OR¹ (IVb′)

may be obtained and/or may be obtainable, wherein, in the general formula (IVb′),
the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and
the radical R² represents one or more of the following radicals

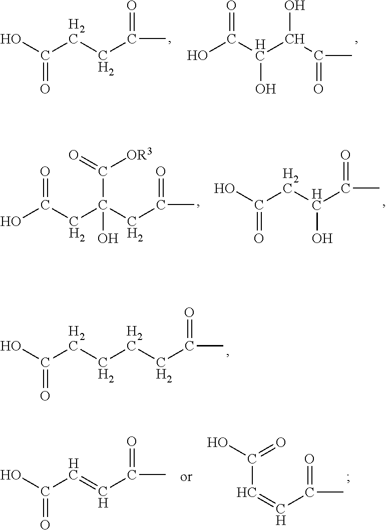

wherein, in the above radicals, the radical R³ represents hydrogen or a radical —CH(CH₃)—CH₂—C(O)OR¹ with R¹ as defined hereinabove.

Moreover, according to the particular embodiment of the method according to the invention, according to which the method is carried out in two steps, as a reaction intermediate product (IV′) of method step (a), a mixture of at least two different polycarboxylic acid esters of oxobutanol, especially polycarboxylic acid esters of 3-hydroxybutanoate, preferably polycarboxylic acid esters of 4-oxo-2-butanol, preferentially polycarboxylic acid esters of 4-oxo-4-(C₁-C₅-alkoxy)-2-butanol or of 4-oxo-4-(hydroxy-C₃-C₅-alkoxy)-2-butanol, especially as defined hereinabove, may be obtained and/or obtainable.

In the method according to the invention, as a reaction product (IV), one or more polyglycerol esters of polycarboxylic acids esterified with oxobutanol, especially one or more polyglycerol esters of polycarboxylic acids esterified with 4-oxo-2-butanol, preferentially one or more mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, of the general formula (IVa)

R⁴O—CH₂13 CH(OR⁴)—CH₂—[O—CH₂—CH(OR⁴)—CH₂]ₚ—OR⁴ (IVa)

may be obtained and/or may be obtainable,

wherein, in the general formula (IVa), the radical R⁴, independently of one other, represents
hydrogen;
a radical R¹—O—C(O)—CH₂—CH(CH₃)—O—(O)C—X—C(O)—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein X represents a saturated or unsaturated and optionally mono- or polysubstituted, especially substituted with one or more hydroxyl radicals and/or radicals —O—C(O)—CH₂—CH(OH)—CH₃ and/or carboxyl radicals and/or radicals —C(O)—O—CH(CH₃)—CH₂—C(O)OR¹ with R¹ as defined hereinabove, organic radical comprising 1 to 10, preferentially comprising 2 to 6 carbon atoms;
a radical H₃C—CH(OH)—CH₂—C(O)—;
a radical HOOC—X—C(O)—, wherein X represents a saturated or unsaturated and optionally mono- or polysubstituted, especially substituted with one or more hydroxyl radicals and/or radicals —O—C(O)—CH₂—CH(OH)—CH₃ and/or carboxyl radicals and/or radicals —C(O)—O—CH(CH₃)—CH₂—C(O)OR¹ substituted with R¹ as defined hereinabove, organic radical comprising 1 to 10, preferentially 2 to 6 carbon atoms;

however, with the proviso that at least one radical R⁴, especially at least two radicals R⁴, represents a radical R¹—O—C(O)—CH₂—CH(CH₃)—O—(O)C—X—C(O)— as defined hereinabove; and

wherein, in the general formula (IVa), the variable p represents an integer from 1 to 6, especially from 1 to 4, preferentially 1 or 2, more preferably 1.

Especially, as a reaction product (IV), one or more polyglycerol esters of polycarboxylic acids esterified with oxobutanol, especially one or more polyglycerol esters of polycarboxylic acids esterified with 4-oxo-2-butanol, preferentially one or more mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, of the general formula (IVa)

R⁴O—CH₂—CH(OR⁴)—CH₂—[O—CH₂—CH(OR⁴)—CH₂]ₚ—OR⁴ (IVa)

may be obtained and/or may be obtainable,

wherein, in the general formula (IVa), the radical R⁴, independently of one another, represents
hydrogen;
a radical R¹—O—C(O)—CH₂—CH(CH₃)—O—(O)C—X—C(O)—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein X represents a saturated or unsaturated and optionally mono- or polysubstituted, especially substituted with one or more hydroxyl radicals and/or radicals —O—C(O)—CH₂—CH(OH)—CH₃ and/or carboxyl radicals and/or radicals —C(O)—O—CH(CH₃)—CH₂—C(O)OR¹ with R¹ as defined hereinabove, organic radical a 1 to 10, preferably 2 to 6 carbon atoms;

however, with the proviso that at least one radical R⁴, especially at least two radicals R⁴, represents a radical R¹—O—C(O)—CH₂—CH(CH₃)—O—(O)C—X—C(O)—, as defined hereinabove; and

wherein, in the general formula (IVa), the variable p represents an integer from 1 to 6, especially from 1 to 4, preferentially 1 or 2, more preferably 1.

According to a particular embodiment of the method according to the invention, it may be preferred if, as a reaction product (IV), one or more polyglycerol esters of polycarboxylic acids esterified with oxobutanol, especially one or more polyglycerol esters of polycarboxylic acids esterified with 4-oxo-2-butanol, preferentially one or more mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, of the general formula (IVa)

R⁴O—CH₂—CH(OR⁴)—CH₂—[O—CH₂—CH(OR⁴)—CH₂]ₚ—OR⁴ (IVa)

are obtained and/or are obtainable,

wherein, in the general formula (IVa), the radical R⁴, independently of one another, represents
hydrogen;
a radical R¹—O—C(O)—CH₂—CH(CH₃)—O—(O)C—X—C(O)—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein X represents a saturated or unsaturated and optionally mono- or polysubstituted, especially substituted with one or more hydroxyl radicals and/or carboxyl radicals, organic radical comprising 1 to 10, preferentially 2 to 6 carbon atoms;

however, with the proviso that at least one radical R⁴, especially at least two radicals R⁴, represents a radical R¹—O—C(O)—CH₂—CH(CH₃)—O—(O)C—X—C(O)—, as defined hereinabove; and

wherein, in the general formula (IVa), the variable p represents an integer from 1 to 6, especially from 1 to 4, preferentially 1 or 2, more preferably 1.

According to a further particular embodiment of the method according to the invention, as a reaction product (IV), one or more polyglycerol esters of polycarboxylic acids esterified with oxobutanol, especially one or more polyglycerol esters of polycarboxylic acids esterified with 4-oxo-2-butanol, preferentially one or more mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, of the general formula (IVb)

R⁵O—CH₂—CH(OR⁵)—CH₂—[O—CH₂—CH(OR⁵)—CH₂]ₚ—OR⁵ (IVb)

may be obtained and/or may be obtainable,

wherein, in the general formula (IVb), the radical R⁵, independently of one another, represents:
hydrogen;
a radical R¹—O—C(O)—CH₂—CH(CH₃)—O—Y—, wherein the radical R¹ represents C₁-C₃-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein Y is derived from a polycarboxylic acid selected from the group of succinic acid, tartaric acid, citric acid, malic acid, adipic acid, fumaric acid and maleic acid as well as combinations or mixtures thereof, especially selected from the group of succinic acid, tartaric acid, citric acid, malic acid, adipic acid and fumaric acid as well as combinations or mixtures thereof, preferably selected from the group of succinic acid and adipic acid and anhydrides thereof as well as combinations or mixtures thereof, especially wherein in the case of citric acid the further carboxyl group present is esterified with a radical —CH(CH₃)—CH₂—C(O)OR¹ with R¹ as defined hereinabove
a radical H₃C—CH(OH)—CH₂—C(O)—;
a radical —Y—OH with Y as defined hereinabove;

however, with the proviso that at least one radical R⁵, especially at least two radicals R⁵, represents a radical R¹—O—C(O)—CH₂—CH(CH₃)—O—Y— with R¹ and Y each as defined hereinabove; and

wherein, in the general formula (IVb), the variable p represents an integer from 1 to 6, especially from 1 to 4, preferentially 1 or 2, more preferably 1.

In this context, “derived from” means that Y is formed from the carboxylic acids mentioned; especially, the hydrogen of the carboxyl is esterified by esterification; i.e. in each case the carboxylate radical of the corresponding acid is present as Y (i.e. Y is a succinate radical in the case of succinic acid, a tartrate radical in the case of tartaric acid, a citrate radical in the case of citric acid, a malate radical in the case of malic acid, an adipate radical in the case of adipic acid, a fumarate radical in the case of fumaric acid and a maleate radical in the case of maleic acid).

In this context, “esterified with” means that in the case of citric acid, in the further, especially third, carboxyl group, the hydrogen is replaced by a radical —CH(CH₃)—CH₂—C(O)OR¹.

According to another particular embodiment of the method according to the invention, as a reaction product (IV), one or more polyglycerol esters of polycarboxylic acids esterified with oxobutanol, especially one or more polyglycerol esters of polycarboxylic acids esterified with 4-oxo-2-butanol, preferentially one or more mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, of the general formula (IVb)

R⁵O—CH₂—CH(OR⁵)—CH₂—[O—CH₂—CH(OR⁵)—CH₂]ₚ—OR (IVb)

may be obtained and/or may be obtainable,

wherein, in the general formula (IVb), the radical R⁵, independently of one another, represents:
hydrogen;
a radical R¹—O—C(O)—CH₂—CH(CH₃)—O—Y—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein Y represents one of the following radicals:

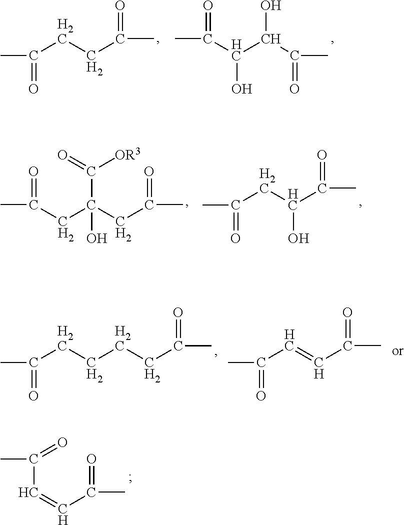

wherein, in the above radicals, the radical R³ represents hydrogen or a radical —CH(CH₃)—CH₂—C(O)OR¹ with R¹ as defined hereinabove;
a radical H₃C—CH(OH)—CH₂—C(O)—;
a radical Y—OH with Y as defined hereinabove;

however, with the proviso that at least one radical R⁵, especially at least two radicals R⁵, represents a radical R¹—O—C(O)—CH₂—CH(CH₃)—O—Y— with R¹ and Y each as defined hereinabove; and

wherein, in the general formula (IVb), the variable p represents an integer from 1 to 6, especially from 1 to 4, preferentially 1 or 2, more preferably 1.

Furthermore, according to a particular embodiment of the method according to the invention, as a reaction product (IV), one or more polyglycerol esters of polycarboxylic acids esterified with oxobutanol, especially one or more polyglycerol esters of polycarboxylic acids esterified with 4-oxo-2-butanol, preferentially one or more mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, of the general formula (IVb)

R⁵O—CH₂—CH(OR⁵)—CH₂—[O—CH₂—CH(OR⁵)—CH₂]ₚ—OR⁵ (IVb)

may be obtained and/or may be obtainable,

wherein, in the general formula (IVb), the radical R⁵, independently of one another, represents:
hydrogen;
a radical R¹—O—C(O)—CH₂—CH(CH₃)—O—Y—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and
   wherein Y represents one of the following radicals:

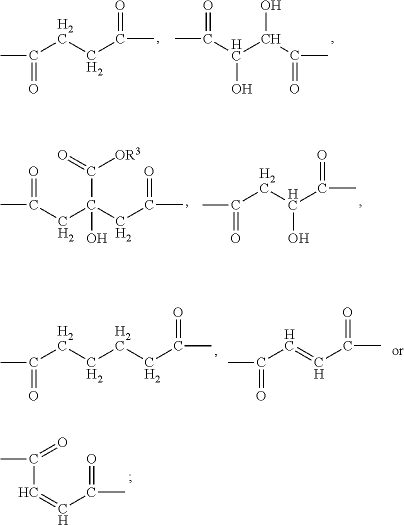

wherein, in the above radicals, the radical R³ represents hydrogen or a radical —CH(CH₃)—CH₂—C(O)OR¹ with R¹ as defined hereinabove;

however, with the proviso that at least one radical R³, especially at least two radicals R⁵, represents a radical R¹—O—C(O)—CH₂—CH(CH₂)—O—Y— with R¹ and Y each as defined hereinabove; and

wherein, in the general formula (IVb), the variable p represents an integer from 1 to 6, especially from 1 to 4, preferentially 1 or 2, more preferably 1.

Moreover, according to a further particular embodiment of the method according to the invention, as a reaction product (IV), one or more polyglycerol esters of polycarboxylic acids esterified with oxobutanol, especially one or more polyglycerol esters of polycarboxylic acids esterified with 4-oxo-2-butanol, preferentially one or more mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, of the general formula (IVc)

R⁴O—CH₂—CH(OR⁴)—CH₂—O—CH₂—CH(OR⁴)—CH₂—OR₄ (IVc)

may be obtained and/or may be obtainable,

wherein, in the general formula (IVc), the radical R⁴, independently of one another, represents:
hydrogen;
a radical R¹—O—C(O)—CH₂—CH(CH₃)—O—(O)C—X—C(O)—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein X represents a saturated or unsaturated and optionally mono- or polysubstituted, especially substituted with one or more hydroxyl radicals and/or radicals —O—C(O)—CH₂—CH(OH)—CH₃ and/or carboxyl radicals and/or radicals —C(O)—O—CH(CH₃)—CH₂—C(O)OR¹ with R¹ as defined hereinabove, organic radical comprising 1 to 10, preferentially 2 to 6 carbon atoms;
a radical H₃C—CH(OH)—CH₂—C(O)—;
a radical HOOC—X—C(O)—, wherein X represents a saturated or unsaturated and optionally mono- or polysubstituted, especially substituted with one or more hydroxyl radicals and/or radicals —O—C(O)—CH₂—CH(OH)—CH₃ and/or carboxyl radicals and/or radicals —C(O)—O—CH(CH₃)—CH₂—C(O)OR¹ substituted with R¹ as defined hereinabove, organic radical comprising 1 to 10, preferentially 2 to 6 carbon atoms;

however, with the proviso that at least one radical R⁴, especially at least two radicals R⁴, represents a radical R¹—O—C(O)—CH₂—CH(CH₃)—O—(O)C—X—C(O)—, as defined hereinabove.

Especially, according to a special embodiment of the method according to the invention, as a reaction product (IV), one or more polyglycerol esters of polycarboxylic acids esterified with oxobutanol, especially one or more polyglycerol esters of polycarboxylic acids esterified with 4-oxo-2-butanol, preferentially one or more mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, of the general formula (IVc)

R⁴O—CH₂—CH(OR⁴)—CH₂—O—CH₂—CH(OR⁴)—CH₂—OR⁴ (IVc)

may be obtained and/or may be obtainable,

wherein, in the general formula (IVc), the radical R⁴, independently of one another, represents:
hydrogen;
a radical R¹—O—C(O)—CH₂—CH(CH₃)—O—(O)C—X—C(O)—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein X represents a saturated or unsaturated and optionally mono- or polysubstituted, especially substituted with one or more hydroxyl radicals and/or radicals —O—C(O)—CH₂—CH(OH)—CH₃ and/or carboxyl radicals and/or radicals —C(O)—O—CH(CH₃)—CH₂—C(O)OR¹ with R¹ as defined hereinabove, organic radical comprising 1 to 10, preferentially 2 to 6 carbon atoms;

however, with the proviso that at least one radical R⁴, especially at least two radicals R⁴, represents a radical R¹—O—C(O)—CH₂—CH(CH₃)—O—(O)C—X—C(O)—, as defined hereinabove.

Furthermore, according to an additional particular embodiment of the method according to the invention, as a reaction product (IV), one or more polyglycerol esters of polycarboxylic acids esterified with oxobutanol, especially one or more polyglycerol esters of polycarboxylic acids esterified with 4-oxo-2-butanol, preferentially one or more mono- or polyvalent polyglycerol esters of polycarboxylic acids esterified with 4-oxo-4-(C₁-C₃-alkoxy)-2-butanol or with 4-oxo-4-(hydroxy-C₃-C₅-alkoxy)-2-butanol, of the general formula (IVc)

R⁴O—CH₂—CH(OR⁴)—CH₂—O—CH₂—CH(OR⁴)—CH₂—OR⁴ (IVc)

may be obtained and/or may be obtainable,

wherein, in the general formula (IVc), the radical R⁴, independently of one another, represents:
hydrogen;
a radical R¹—O—C(O)—CH₂—CH(CH₃)—O—(O)C—X—C(O)—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein X represents a saturated or unsaturated and optionally mono- or polysubstituted, especially substituted with one or more hydroxyl radicals and/or carboxyl radicals, organic radical comprising 1 to 10, preferentially 2 to 6 carbon atoms;

however, with the proviso that at least one radical R⁴, especially at least two radicals R⁴, represents a radical R¹—O—C(O)—CH₂—CH(CH₃)—O—(O)C—X—C(O)—, as defined hereinabove.

Especially, according to a special embodiment of the method according to the invention, as a reaction product (IV), one or more polyglycerol esters of polycarboxylic acids esterified with oxobutanol, especially one or more polyglycerol esters of polycarboxylic acids esterified with 4-oxo-2-butanol, preferentially one or more mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, of the general formula (IVd)

R⁵O—CH₂—CH(OR⁵)—CH₂—O—CH₂—CH(OR⁵)—CH₂—OR⁵ (IVd)

may be obtained and/or may be obtainable,

wherein, in the general formula (IVd), the radical R³, independently of one another, represents:
hydrogen;
a radical R¹—O—C(O)—CH₂—CH(CH₃)—O—Y—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein Y is derived from a polycarboxylic acid selected from the group of succinic acid, tartaric acid, citric acid, malic acid, adipic acid, fumaric acid and maleic acid as well as combinations or mixtures thereof, especially selected from the group of succinic acid, tartaric acid, citric acid, malic acid, adipic acid and fumaric acid as well as combinations or mixtures thereof, preferably selected from the group of succinic acid and adipic acid and anhydrides thereof as well as combinations or mixtures thereof, especially wherein, in the case of citric acid, the further carboxyl group present is esterified with a radical —CH(CH₃)—CH₂—C(O)OR¹ with R¹ as defined hereinabove
a radical H₃C—CH(OH)—CH₂—C(O)—;
a radical Y—OH with Y as defined hereinabove;

however, with the proviso that at least one radical R³, especially at least two radicals R⁵, represents a radical R¹—O—C(O)—CH₂—CH(CH₃)—O—Y— with R¹ and Y each as defined hereinabove.

In this context, “derived from” means that Y is formed from the carboxylic acids mentioned; especially, the hydrogen of the carboxyl is esterified by esterification; i.e. in each case the carboxylate radical of the corresponding acid is present as Y (i.e. Y is a succinate radical in the case of succinic acid, a tartrate radical in the case of tartaric acid, a citrate radical in the case of citric acid, a malate radical in the case of malic acid, an adipate radical in the case of adipic acid, a fumarate radical in the case of fumaric acid and a maleate radical in the case of maleic acid).

In this context, “esterified with” means that in the case of citric acid, the hydrogen of the further, especially third, carboxyl group is replaced by a radical —CH(CH₃)—CH₂—C(O)OR¹.

Furthermore, according to a particular embodiment of the method according to the invention, as a reaction product (IV), one or more polyglycerol esters of polycarboxylic acids esterified with oxobutanol, especially one or more polyglycerol esters of polycarboxylic acids esterified with 4-oxo-2-butanol, preferentially one or more mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, of the general formula (IVd)

R⁵O—CH₂—CH(OR⁵)—CH₂—O—CH₂—CH(OR⁵)—CH₂—OR⁵ (IVd)

may be obtained and/or may be obtainable,

wherein, in the general formula (IVd), the radical R³, independently of one another, represents:
hydrogen;
a radical R¹—O—C(O)—CH₂—CH(CH₃)—O—Y—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein Y represents one of the following radicals

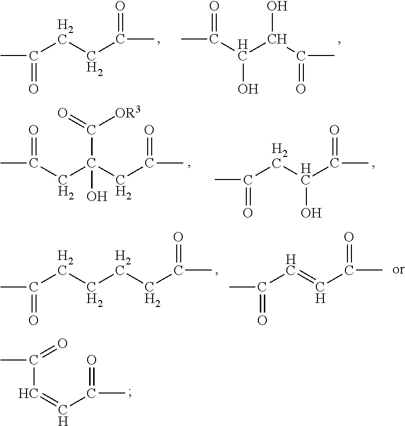

wherein, in the above radicals, the radical R³ represents hydrogen or a radical —CH(CH₃)—CH₂—C(O)OR¹ with R¹ as defined hereinabove;
a radical H₃C—CH(OH)—CH₂—C(O)—;
a radical Y—OH with Y as defined hereinabove;

however, with the proviso that at least one radical R⁵, especially at least two radicals R⁵, represents a radical R¹—O—C(O)—CH₂—CH(CH₃)—O—Y— with R¹ and Y each as defined hereinabove.

Furthermore, according to a particular embodiment of the inventive method, as a reaction product (IV), one or more polyglycerol esters of polycarboxylic acids esterified with oxobutanol, especially one or more polyglycerol esters of polycarboxylic acids esterified with 4-oxo-2-butanol, preferentially one or more mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, of the general formula (IVd)

R⁵O—CH₂—CH(OR⁵)—CH₂—O—CH₂—CH(OR⁵)—CH₂—OR⁵ (IVd)

may be obtained and/or may be obtainable,

wherein, in the general formula (IVd), the radical R⁵, independently of one another, represents:
hydrogen;
a radical R¹—O—C(O)—CH₂—CH(CH₃)—O—Y—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein Y represents one of the following radicals

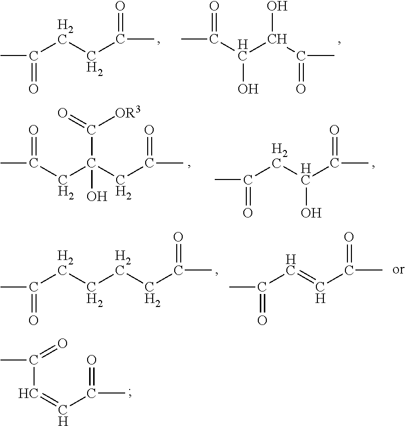

wherein, in the above radicals, the radical R³ represents hydrogen or a radical —CH(CH₃)—CH₂—C(O)OR¹ with R¹ as defined hereinabove;

however, with the proviso that at least one radical R⁵, especially at least two radicals R⁵, represents a radical R¹—O—C(O)—CH₂—CH(CH₃)—O—Y— with R¹ and Y each as defined hereinabove.

According to another particular embodiment of the method according to the invention, as a reaction product (IV), a mixture of at least two different polyglycerol esters of polycarboxylic acids esterified with oxobutanol, especially polyglycerol esters of polycarboxylic acids esterified with 4-oxo-2-butanol, preferentially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, may be obtained and/or may be obtainable.

A further subject-matter—according to a second aspect of the present invention—is a reaction intermediate product or a polycarboxylic acid ester of oxobutanol, especially polycarboxylic acid ester of 3-hydroxybutanoate, preferably polycarboxylic acid ester of 4-oxo-2-butanol, preferentially polycarboxylic acid ester of 4-oxo-4-(C₁-C₅-alkoxy)-2-butanol or of 4-oxo-4-(hydroxy-C₃-C₅-alkoxy)-2-butanol, obtainable by a method described hereinabove, especially obtainable as reaction intermediate product (IV′) of the first method step (a) of the method described hereinabove.

According to a particular embodiment, the reaction intermediate product may especially comprise a mixture comprising at least two different polycarboxylic acid esters of oxobutanol, as previously defined.

However, it is also an object of the present invention according to this aspect of the invention to provide a reaction product or a polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, obtainable by the method as described hereinabove.

According to a particular embodiment, the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, according to the invention or obtainable according to the inventive method, may correspond to the general formula (IVa)

R⁴O—CH₂—CH(OR⁴)—CH₂—[O—CH₂—CH(OR⁴)—CH₂]ₚ—OR⁴ (IVa)

wherein, in the general formula (IVa), the radical R⁴, independently of one another, represents:
hydrogen;
a radical R¹—O—C(O)—CH₂—CH(CH₃)—O—(O)C—X—C(O)—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein X represents a saturated or unsaturated and optionally mono- or polysubstituted, especially substituted with one or more hydroxyl radicals and/or radicals —O—C(O)—CH₂—CH(OH)—CH₃ and/or carboxyl radicals and/or radicals —C(O)—O—CH(CH₃)—CH₂—C(O)OR¹ with R¹ as defined hereinabove, organic radical comprising 1 to 10, preferentially comprising 2 to 6 carbon atoms;
a radical H₃C—CH(OH)—CH₂—C(O)—;
a radical HOOC—X—C(O)—, wherein X represents a saturated or unsaturated and optionally mono- or polysubstituted, especially substituted with one or more hydroxyl radicals and/or radicals —O—C(O)—CH₂—CH(OH)—CH₃ and/or carboxyl radicals and/or radicals —C(O)—O—CH(CH₃)—CH₂—C(O)OR¹ substituted with R¹ as defined hereinabove, organic radical comprising 1 to 10, preferentially 2 to 6 carbon atoms;

however, with the proviso that at least one radical R⁴, especially at least two radicals R⁴, represents a radical R¹—O—C(O)—CH₂—CH(CH₂)—O—(O)C—X—C(O)—, as defined hereinabove; and

wherein, in the general formula (IVa), the variable p represents an integer from 1 to 6, especially from 1 to 4, preferentially 1 or 2, more preferably 1.

According to another particular embodiment, the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, according to the invention or obtainable according to the inventive method, may correspond to the general formula (IVa)

R⁴O—CH₂—CH(OR⁴)—CH₂—[O—CH₂—CH(OR⁴)—CH₂]ₚ—OR⁴ (IVa)

wherein, in the general formula (IVa), the radical R⁴, independently of one another, represents:
hydrogen;
a radical R¹—O—C(O)—CH₂—CH(CH₃)—O—(O)C—X—C(O)—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein X represents a saturated or unsaturated and optionally mono- or polysubstituted, especially substituted with one or more hydroxyl radicals and/or radicals —O—C(O)—CH₂—CH(OH)—CH₃, and/or carboxyl radicals and/or radicals —C(O)—O—CH(CH₃)—CH₂—C(O)OR¹ with R¹ as defined hereinabove, organic radical comprising 1 to 10, preferentially 2 to 6 carbon atoms;

however, with the proviso that at least one radical R⁴, especially at least two radicals R⁴, represents a radical R¹—O—C(O)—CH₂—CH(CH₃)—O—(O)C—X—C(O)—, as defined hereinabove; and

wherein, in the general formula (IVa), the variable p represents an integer from 1 to 6, especially from 1 to 4, preferentially 1 or 2, more preferably 1.

According to a further particular embodiment of the present invention, the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, according to the invention or obtainable according to the inventive method, may correspond to the general formula (IVa)

R⁴O—CH₂—CH(OR⁴)—CH₂—[O—CH₂—CH(OR⁴)—CH₂]ₚ—OR⁴ (IVa)

wherein, in the general formula (IVa), the radical R⁴, independently of one another, represents:
hydrogen;
a radical R¹—O—C(O)—CH₂—CH(CH₃)—O—(O)C—X—C(O)—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein X represents a saturated or unsaturated and optionally mono- or polysubstituted, especially substituted with one or more hydroxyl radicals and/or carboxyl radicals, organic radical comprising 1 to 10, preferentially 2 to 6 carbon atoms;

however, with the proviso that at least one radical R⁴, especially at least two radicals R⁴, represents a radical R¹—O—C(O)—CH₂—CH(CH₃)—O—(O)C—X—C(O)—, as defined hereinabove; and

wherein, in the general formula (IVa), the variable p represents an integer from 1 to 6, especially from 1 to 4, preferentially 1 or 2, more preferably 1.

According to a further particular embodiment of the present invention, the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, according to the invention or obtainable according to the inventive method, may correspond to the general formula (IVb)

R⁵O—CH₂—CH(OR⁵)—CH₂—[O—CH₂—CH(OR⁵)—CH₂]ₚ—OR⁵ (IVb)

wherein, in the general formula (IVb), the radical R⁵, independently of one another, represents:
hydrogen;
a radical R¹—O—C(O)—CH₂—CH(CH₃)—O—Y—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein Y is derived from a polycarboxylic acid selected from the group of succinic acid, tartaric acid, citric acid, malic acid, adipic acid, fumaric acid and maleic acid as well as combinations or mixtures thereof, especially selected from the group of succinic acid, tartaric acid, citric acid, malic acid, adipic acid and fumaric acid as well as combinations or mixtures thereof, preferably selected from the group of succinic acid and adipic acid and anhydrides thereof as well as combinations or mixtures thereof, especially wherein in the case of citric acid the further carboxyl group present is esterified with a radical —CH(CH₃)—CH₂—C(O)OR¹ with R¹ as defined hereinabove
a radical H₃C—CH(OH)—CH₂—C(O)—;
a radical —Y—OH with Y as defined hereinabove;

however, with the proviso that at least one radical R⁵, especially at least two radicals R⁵, represents a radical R¹—O—C(O)—CH₂—CH(CH₃)—O—Y— with R¹ and Y each as defined hereinabove; and

wherein, in the general formula (IVb), the variable p represents an integer from 1 to 6, especially from 1 to 4, preferentially 1 or 2, more preferably 1.

In this context, “derived from” means that Y is formed from the carboxylic acids mentioned; especially, the hydrogen of the carboxyl is esterified by esterification; i.e. in each case the carboxylate radical of the corresponding acid is present as Y (i.e. Y is a succinate radical in the case of succinic acid, a tartrate radical in the case of tartaric acid, a citrate radical in the case of citric acid, a malate radical in the case of malic acid, an adipate radical in the case of adipic acid, a fumarate radical in the case of fumaric acid and a maleate radical in the case of maleic acid).

In this context, “esterified with” means that in the case of citric acid, in the further, especially third, carboxyl group, the hydrogen is replaced by a radical —CH(CH₃)—CH₂—C(O)OR¹.

According to a preferred embodiment of the present invention, the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, according to the invention or obtainable according to the inventive method, may correspond to the general formula (IVb)

R⁵O—CH₂—CH(OR⁵)—CH₂—[O—CH₂—CH(OR⁵)—CH₂]ₚ—OR⁵ (IVb)

wherein, in the general formula (IVb), the radical R⁵, independently of one another, represents:
hydrogen;
a radical R¹—O—C(O)—CH₂—CH(CH₃)—O—Y—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein Y represents one of the following radicals:

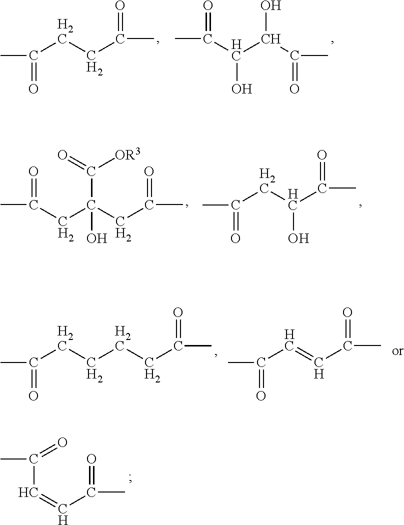

wherein, in the above radicals, the radical R³ represents hydrogen or a radical —CH(CH₃)—CH₂—C(O)OR¹ with R³ as defined hereinabove;
a radical H₃C—CH(OH)—CH₂—C(O)—;
a radical Y—OH with Y as defined hereinabove;

however, with the proviso that at least one radical R³, especially at least two radicals R⁵, represents a radical R¹—O—C(O)—CH₂—CH(CH₃)—O—Y— with R¹ and Y each as defined hereinabove; and

wherein, in the general formula (IVb), the variable p represents an integer from 1 to 6, especially from 1 to 4, preferentially 1 or 2, more preferably 1.

According to a further preferred embodiment of this aspect of the invention, the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, according to the invention or obtainable according to the inventive method, may correspond to the general formula (IVb)

R⁵O—CH₂—CH(OR⁵)—CH₂—[O—CH₂—CH(OR⁵)—CH₂]ₚ—OR⁵ (IVb)

wherein, in the general formula (IVb), the radical R⁵, independently of one another, represents:
hydrogen;
a radical R¹—O—C(O)—CH₂—CH(CH₃)—O—Y—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein Y represents one of the following radicals:

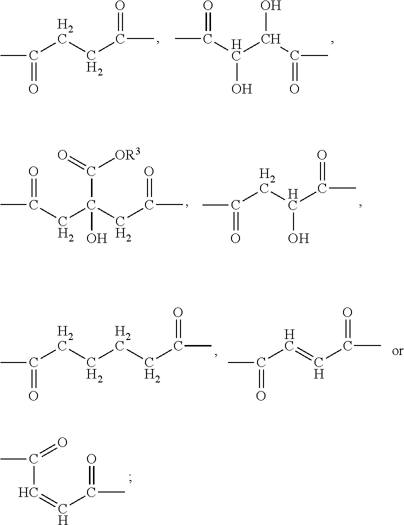

wherein, in the above radicals, the radical R³ represents hydrogen or a radical —CH(CH₃)—CH₂—C(O)OR¹ with R¹ as defined hereinabove;

however, with the proviso that at least one radical R⁵, especially at least two radicals R⁵, represents a radical R¹—O—C(O)—CH₂—CH(CH₃)—O—Y— with R¹ and Y each as defined hereinabove; and

wherein, in the general formula (IVb), the variable p represents an integer from 1 to 6, especially from 1 to 4, preferentially 1 or 2, more preferably 1.

Furthermore, according to a particular embodiment of this aspect of the invention, the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, according to the invention or obtainable according to the inventive method, may correspond to the general formula (IVc)

R⁴O—CH₂—CH(OR⁴)—CH₂—O—CH₂—CH(OR⁴)—CH₂—OR⁴ (IVc)

wherein, in the general formula (IVc), the radical R⁴, independently of one another, represents
hydrogen;
a radical R¹—O—C(O)—CH₂—CH(CH₃)—O—(O)C—X—C(O)—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein X represents a saturated or unsaturated and optionally mono- or polysubstituted, especially substituted with one or more hydroxyl radicals and/or radicals —O—C(O)—CH₂—CH(OH)—CH₃ and/or carboxyl radicals and/or radicals —C(O)—O—CH(CH₃)—CH₂—C(O)OR¹ with R¹ as defined hereinabove, organic radical comprising 1 to 10, preferentially comprising 2 to 6 carbon atoms;
a radical H₃C—CH(OH)—CH₂—C(O)—;
a radical HOOC—X—C(O)—, wherein X represents a saturated or unsaturated and optionally mono- or polysubstituted, especially substituted with one or more hydroxyl radicals and/or radicals —O—C(O)—CH₂—CH(OH)—CH₃ and/or carboxyl radicals and/or radicals —C(O)—O—CH(CH₃)—CH₂—C(O)OR¹ substituted with R¹ as defined hereinabove, organic radical comprising 1 to 10, preferentially 2 to 6 carbon atoms;

however, with the proviso that at least one radical R⁴, especially at least two radicals R⁴, represents a radical R¹—O—C(O)—CH₂—CH(CH₃)—O—(O)C—X—C(O)—, as defined hereinabove.

Furthermore, according to a further particular embodiment of this aspect of the invention, the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, according to the invention or obtainable according to the inventive method, may correspond to the general formula (IVc)

R⁴O—CH₂—CH(OR⁴)—CH₂—O—CH₂—CH(OR⁴)—CH₂—OR⁴ (IVc)

wherein, in the general formula (IVc), the radical R⁴, independently of one another, represents
hydrogen;
a radical R¹—O—C(O)—CH₂—CH(CH₃)—O—(O)C—X—C(O)—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein X represents a saturated or unsaturated and optionally mono- or polysubstituted, especially substituted with one or more hydroxyl radicals and/or radicals —O—C(O)—CH₂—CH(OH)—CH₃ and/or carboxyl radicals and/or radicals —C(O)—O—CH(CH₃)—CH₂—C(O)OR¹ with R¹ as defined hereinabove, organic radical comprising 1 to 10, preferentially 2 to 6 carbon atoms;

however, with the proviso that at least one radical R⁴, especially at least two radicals R⁴, represents a radical R¹—O—C(O)—CH₂—CH(CH₃)—O—(O)C—X—C(O)—, as defined hereinabove.

Also, according to yet another particular embodiment of this aspect of the invention, the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, according to the invention or obtainable according to the inventive method, may correspond to the general formula (IVc)

R⁴O—CH₂—CH(OR⁴)—CH₂—O—CH₂—CH(OR⁴)—CH₂—OR⁴ (IVc)

wherein, in the general formula (IVc), the radical R⁴, independently of one another, represents
hydrogen;
a radical R¹—O—C(O)—CH₂—CH(CH₃)—O—(O)C—X—C(O)—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein X represents a saturated or unsaturated and optionally mono- or polysubstituted, especially substituted with one or more hydroxyl radicals and/or carboxyl radicals organic radical comprising 1 to 10, preferably 2 to 6 carbon atoms;

however, with the proviso that at least one radical R⁴, especially at least two radicals R⁴, represents a radical R¹—O—C(O)—CH₂—CH(CH₃)—O—(O)C—X—C(O)—, as defined hereinabove.

Furthermore, according to a particular embodiment of this aspect of the invention, it can be provided that the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, according to the invention or obtainable according to the inventive method, corresponds to the general formula (IVd)

R⁵O—CH₂—CH(OR⁵)—CH₂—O—CH₂—CH(OR⁵)—CH₂—OR⁵ (IVd)

wherein, in the general formula (IVd), the radical R⁵, independently of one another, represents
hydrogen;
a radical R¹—O—C(O)—CH₂—CH(CH₃)—O—Y—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein Y is derived from a polycarboxylic acid selected from the group of succinic acid, tartaric acid, citric acid, malic acid, adipic acid, fumaric acid and maleic acid as well as combinations or mixtures thereof, especially selected from the group of succinic acid, tartaric acid, citric acid, malic acid, adipic acid and fumaric acid as well as combinations or mixtures thereof, preferably selected from the group of succinic acid and adipic acid and anhydrides thereof as well as combinations or mixtures thereof, especially wherein in the case of citric acid the further carboxyl group present is esterified with a radical —CH(CH₃)—CH₂—C(O)O R¹ with R¹ as defined hereinabove
a radical H₃C—CH(OH)—CH₂—C(O)—;
a radical Y—OH with Y as defined hereinabove;

however, with the proviso that at least one radical R⁵, especially at least two radicals R⁵, represents a radical R¹—O—C(O)—CH₂—CH(CH₃)—O—Y— with R¹ and Y each as defined hereinabove.

In this context, “derived from” means that Y is formed from the carboxylic acids mentioned; especially, the hydrogen of the carboxyl is esterified by esterification; i.e. in each case the carboxylate radical of the corresponding acid is present as Y (i.e. Y is a succinate radical in the case of succinic acid, a tartrate radical in the case of tartaric acid, a citrate radical in the case of citric acid, a malate radical in the case of malic acid, an adipate radical in the case of adipic acid, a fumarate radical in the case of fumaric acid and a maleate radical in the case of maleic acid).

In this context, “esterified with” means that in the case of citric acids in the further, especially third, carboxyl group, the hydrogen is replaced by a radical —CH(CH₃)—CH₂—C(O)OR¹.

Also, according to a further particular embodiment of this aspect of the invention, it can be provided that the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, according to the invention or obtainable according to the inventive method, corresponds to the general formula (IVd)

R⁵O—CH₂—CH(OR⁵)—CH₂—O—CH₂—CH(OR⁵)—CH₂—OR⁵ (IVd)

wherein, in the general formula (IVd), the radical R⁵, independently of one another, represents
hydrogen;
a radical R¹—O—C(O)—CH₂—CH(CH₃)—O—Y—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein Y represents one of the following radicals

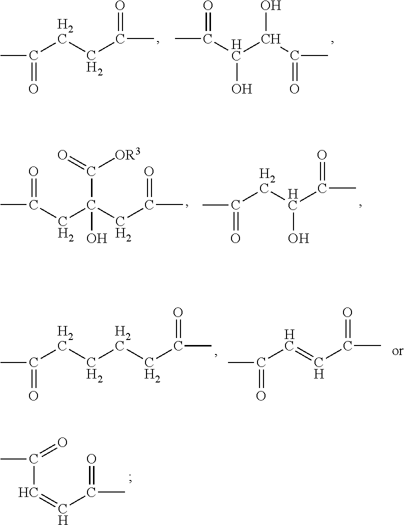

wherein, in the above radicals, the radical R³ represents hydrogen or a radical —CH(CH₃)—CH₂—C(O)OR¹ with R¹ as defined hereinabove;
a radical H₃C—CH(OH)—CH₂—C(O)—;
a radical Y—OH with Y as defined hereinabove;
   however, with the proviso that at least one radical R⁵, especially at least two radicals R⁵, represents a radical R¹—O—C(O)—CH₂—CH(CH₃)—O—Y— with R¹ and Y each as defined hereinabove.

Furthermore, according to a particular embodiment of this aspect of the invention, the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, according to the invention or obtainable according to the inventive method, may correspond to the general formula (IVd)

R⁵O—CH₂—CH(OR⁵)—CH₂—O—CH₂—CH(OR⁵)—CH₂—OR⁵ (IVd)

wherein, in the general formula (IVd), the radical R⁵, independently of one another, represents
hydrogen;
a radical R¹—O—C(O)—CH₂—CH(CH₃)—O—Y—, wherein the radical R¹ represents C₁-C₅-alkyl or hydroxy-C₃-C₅-alkyl, especially ethyl, butyl, pentyl, hydroxybutyl or hydroxypentyl, preferably ethyl, hydroxybutyl or hydroxypentyl, more preferably ethyl, and wherein Y represents one of the following radicals

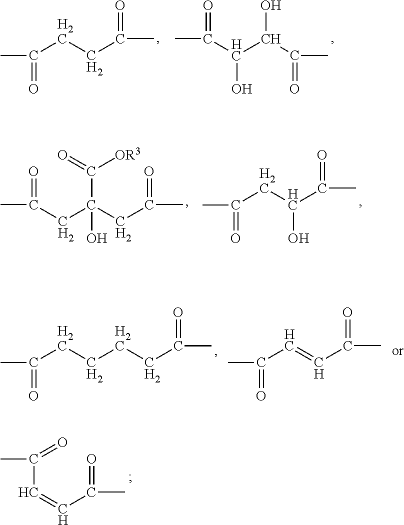

wherein, in the above radicals, the radical R³ represents hydrogen or a radical —CH(CH₃)—CH₂—C(O)OR¹ with R¹ as defined hereinabove;

however, with the proviso that at least one radical R⁵, especially at least two radicals R⁵, represents a radical R¹—O—C(O)—CH₂—CH(CH₃)—O—Y— with R¹ and Y each as defined hereinabove.

Furthermore, according to a particular embodiment of this aspect of the invention, a further object of the present invention is an inventive mixture comprising at least two different polyglycerol esters of polycarboxylic acids esterified with oxobutanol, especially polyglycerol esters of polycarboxylic acids esterified with 4-oxo-2-butanol, preferentially mono- or polyvalent polyglycerol esters of polycarboxylic acids esterified with 4-oxo-4-(C₁-C₅-alkoxy)-2-butanol or with 4-oxo-4-(hydroxy-C₃-C₅-alkoxy)-2-butanol, especially as defined hereinabove.

The reaction product obtainable according to the inventive method or the inventive reaction product as defined hereinabove, respectively, and/or the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, obtainable according to the inventive production method or the inventive polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, as defined hereinabove, respectively, and/or the mixture, obtainable according to the inventive production method or the inventive mixture as defined hereinabove, respectively, comprises a multitude of advantages and special features compared to the prior art:

As the applicant has surprisingly found out, the reaction product obtainable according to the inventive method or the inventive reaction product as defined hereinabove, respectively, and/or the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, obtainable according to the inventive production method or the inventive polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, as defined hereinabove, respectively, and/or the mixture, obtainable according to the inventive production method or the inventive mixture as defined hereinabove, respectively, is suitable as a precursor or metabolite of 3-hydroxybutyric acid or its salts, since, on the one hand, it is converted physiologically, especially in the gastrointestinal tract, to 3-hydroxybutyric acid or its salts and, on the other hand, it simultaneously comprises a good physiological compatibility or tolerability, especially with regard to non-toxicity and acceptable organoleptic properties.

Moreover, the reaction product obtainable according to the inventive method or the inventive reaction product as defined hereinabove, respectively, and/or the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, obtainable according to the inventive production method or the inventive polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, as defined hereinabove, respectively, and/or the mixture, obtainable according to the inventive production method or the inventive mixture as defined hereinabove, respectively, is easily accessible or available on a large scale on a synthetic basis, even on a commercial scale, and with the required pharmaceutical or pharmacological quality.

Additionally, the reaction product obtainable according to the inventive method or the inventive reaction product as defined hereinabove, respectively, and/or the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, obtainable according to the inventive production method or the inventive polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, as defined hereinabove, respectively, and/or the mixture, obtainable according to the inventive production method or the inventive mixture as defined hereinabove, respectively, can, if necessary, be provided in enantiomerically pure or enantiomerically enriched form.

The reaction product obtainable according to the inventive method or the inventive reaction product as defined hereinabove, respectively, and/or the polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, obtainable according to the inventive production method or the inventive polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, as defined hereinabove, respectively, and/or the mixture, obtainable according to the inventive production method or the inventive mixture as defined hereinabove, respectively, thus represents an efficient pharmacological drug target in the context of keto-body therapy of the human or animal body.

In the following, the remaining aspects of the invention are explained in more detail.

A further subject-matter of the present invention—according to a third aspect of the present invention—is a pharmaceutical composition, especially a drug or medicament, which comprises a reaction product obtainable according to the inventive production method or the inventive reaction product, respectively, and/or a polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, obtainable according to the inventive production method or the inventive polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, as defined hereinabove, respectively, and/or a mixture, obtainable according to the inventive production method or the inventive mixture as defined hereinabove, respectively.

Especially, according to this aspect of the invention, the present invention relates to a pharmaceutical composition for the prophylactic and/or therapeutic treatment or for use in the prophylactic and/or therapeutic treatment of diseases of the human or animal body. This may especially concern diseases associated with a disorder of the energy metabolism, especially keto-body metabolism, such as especially craniocerebral trauma, stroke, hypoxia, cardiovascular diseases such as myocardial infarction, refeeding syndrome, anorexia, epilepsy, neurodegenerative diseases such as dementia, Alzheimer's disease, Parkinson's disease, multiple sclerosis and amyotrophic lateral sclerosis, fat metabolic diseases such as glucose transporter defect (GLUT1 defect), VL-FAOD and mitochondriopathies such as mitochondrial thiolase defect, Huntington's disease, cancers such as T-cell lymphomas, astrocytomas and glioblastomas, HIV, rheumatic diseases such as rheumatoid arthritis and arthritis urica, diseases of the gastrointestinal tract such as chronic inflammatory bowel diseases, especially ulcerative colitis and Crohn's disease, lyosomal storage diseases such as sphingolipidosis, especially Niemann-Pick disease, diabetes mellitus and effects or side-effects of chemotherapy.

Again, a further subject-matter of the present invention—according to a fourth aspect of the present invention—is a reaction product obtainable according to the inventive production method or the inventive reaction product, respectively, or a polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, obtainable according to the inventive production method or the inventive polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, as defined hereinabove, respectively, and/or a mixture, obtainable according to the inventive production method or the inventive mixture as defined hereinabove, respectively, for the prophylactic and/or therapeutic treatment or for use in the prophylactic and/or therapeutic treatment of diseases of the human or animal body, especially diseases associated with a disorder of the energy metabolism, especially keto-body metabolism, such as especially craniocerebral trauma, stroke, hypoxia, cardiovascular diseases such as myocardial infarction, refeeding syndrome, anorexia, epilepsy, neurodegenerative diseases such as dementia, Alzheimer's disease, Parkinson's disease, multiple sclerosis and amyotrophic lateral sclerosis, fat metabolic diseases such as glucose transporter defect (GLUT1 defect), VL-FAOD and mitochondriopathies such as mitochondrial thiolase defect, Huntington's disease, cancers such as T-cell lymphomas, astrocytomas and glioblastomas, HIV, rheumatic diseases such as rheumatoid arthritis and arthritis urica, diseases of the gastrointestinal tract such as chronic inflammatory bowel diseases, especially ulcerative colitis and Crohn's disease, lyosomal storage diseases such as sphingolipidosis, especially Niemann-Pick disease, diabetes mellitus and effects or side-effects of chemotherapy.

Likewise, a further subject-matter of the present invention—according to a fifth aspect of the present invention—is the use of a reaction product obtainable according to the inventive production method or the inventive reaction product, respectively, or a polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, obtainable according to the inventive production method or the inventive polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, as defined hereinabove, respectively, and/or a mixture, obtainable according to the inventive production method or the inventive mixture as defined hereinabove, respectively, for the prophylactic and/or therapeutic treatment or for producing a pharmaceutical for the prophylactic and/or therapeutic treatment of diseases of the human or animal body, especially diseases associated with a disorder of the energy metabolism, especially keto-body metabolism, such as especially craniocerebral trauma, stroke, hypoxia, cardiovascular diseases such as myocardial infarction, refeeding syndrome, anorexia, epilepsy, neurodegenerative diseases such as dementia, Alzheimer's disease, Parkinson's disease, multiple sclerosis and amyotrophic lateral sclerosis, fat metabolic diseases such as glucose transporter defect (GLUT1 defect), VL-FAOD and mitochondriopathies such as mitochondrial thiolase defect, Huntington's disease, cancers such as T-cell lymphomas, astrocytomas and glioblastomas, HIV, rheumatic diseases such as rheumatoid arthritis and arthritis urica, diseases of the gastrointestinal tract such as chronic inflammatory bowel diseases, especially ulcerative colitis and Crohn's disease, lyosomal storage diseases such as sphingolipidosis, especially Niemann-Pick disease, diabetes mellitus and effects or side-effects of chemotherapy.

Likewise, a further subject-matter of the present invention—according to a sixth aspect of the present invention—is the use of a reaction product obtainable according to the inventive production method or the inventive reaction product, respectively, or a polyglycerol ester of polycarboxylic acids esterified with oxobutanol, especially polyglycerol ester of polycarboxylic acids esterifed with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, obtainable according to the inventive production method or the inventive polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, as defined hereinabove, respectively, and/or a mixture, obtainable according to the inventive production method or the inventive mixture as defined hereinabove, respectively, for the prophylactic and/or therapeutic treatment or for producing a medicament for the prophylactic and/or therapeutic treatment of or for the application for catabolic metabolic states, such as hunger, diets or low-carbohydrate nutrition.

Likewise, a further subject-matter of the present invention—according to a seventh aspect of the present invention—is a food and/or a food product, which comprises a reaction product obtainable according to the inventive production method or the inventive reaction product, respectively, or a polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₁-C₅-alkoxy)-2-butanol, obtainable according to the inventive production method or the inventive polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, as defined hereinabove, respectively, and/or a mixture, obtainable according to the inventive production method or the inventive mixture as defined hereinabove, respectively.

According to a particular embodiment, the food and/or the food product may essentially be a dietary supplement, a functional food, a novel food, a food additive, a food supplement, a dietary food, a power snack, an appetite suppressant or a strength and/or endurance sport supplement.

Finally, yet another subject-matter of the present invention—according to an eighth aspect of the present invention—is the use of a reaction product obtainable according to the inventive production method or a polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, obtainable according to the inventive production method or the inventive polyglycerol ester of polycarboxylic acids esterified with oxobutanol, especially polyglycerol ester of polycarboxylic acids esterified with 4-oxo-2-butanol, preferentially mono- or polyvalent polyglycerol ester of polycarboxylic acids esterified with 4-oxo-4-(C₁-C₅-alkoxy)-2-butanol or with 4-oxo-4-(hydroxy-C₃-C₅-alkoxy)-2-butanol, as defined hereinabove, respectively, and/or a mixture, obtainable according to the inventive production method as defined hereinabove, respectively, in a food and/or a food product.

According to this aspect of the invention, the food and/or the food product may especially be a dietary supplement, a functional food, a novel food, a food additive, a food supplement, a dietary food, a power snack, an appetite suppressant or a strength and/or endurance sports supplement.

Further embodiments, modifications and variations of the present invention are readily recognizable or realizable by a person skilled in the art when reading the description, without leaving the scope of the present invention.

The present invention is illustrated by the following examples, which are not intended to limit the present invention in any way, but only to explain the exemplary and non-limiting implementation and configuration of the present invention.

### EXAMPLES

### Abbreviations Used

| | |
|---|---|
| 3-BHB-EE = BHB-EE | 3-hydroxybulyric acid ethyl ester (=ethyl |
| | 3-hydroxybutanoate or 4-ethoxy-4-oxobutan-2-ol) |
| 3-BHB = BHB | 3-hydroxybutyric add |
| BS-(BHB-EE)monoester | succinic acid monoester of ethyl 3-hydroxy- |
| | butanoate |
| BS-(BHB-EE)monoester | succinic diester of ethyl 3-hydroxybutanoate |
| PG(2)-(BS-(BHB-EE))monoester | diglycerol monoester of succinic acid esterified with |
| | ethyl 3-hydroxybutanoate |
| PG(2)-(BS-(BHB-EE))diester | diglycerol diester of succinic acid esterified with ethyl |
| | 3-hydroxybutanoate |
| PG(2)-(BS-(BHB-EE))polyester | diglycerol polyester of succinic acid esterified with |
| | ethyl 3-hydroxybutanoate |

### I. Production Examples of a Particular Embodiment of the Method According to the Invention as a One-Pot Synthesis without the Use of a Catalyst

### I. 1. Production of Digylcerol Esters of Succinic Acid Esterified with Ethyl 3-hydroxybutanoate Using Succinic Acid Anhydride

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic), 100 g succinic acid anhydride and 40 g diglycerol are provided. The reaction mixture is reacted with stirring at 120° C. and under N₂ for 7 h, and the resulting reaction water is continuously withdrawn by distillation. Subsequently, excess ethyl 3-hydroxybutyric acid ester and excess succinic acid ester of ethyl 3-hydroxybutanoate are withdrawn by distillation under vacuum. The desired reaction product mixture is then obtained as a residue. The residue obtained is steam treated in a vacuum for 2 to 4 hours if required.

A mixture of mono-, di-, tri- and tetravalent esters of diglycerol (i.e. diglycerol monoester, diglycerol diester, diglycerol triester and diglycerol tetraester) of succinic acid esterified with ethyl 3-hydroxybutanoate is obtained (see also reaction scheme below).

By-products formed include very small amounts of diglycerol esters of 3-hydroxybutyric acid and diglycerol esters of succinic acid.

The diglycerol monoester of succinic acid esterified with ethyl 3-hydroxybutanoate may also be referred to synonymously as 1-[3-(2,3-dihydroxypropoxy)-2-hydroxypropyl] 4-(4-ethoxy-4-oxobutan-2-yl)-butanedioate.

The diglycerol diester of succinic acid esterified with ethyl 3-hydroxybutanoate may also be referred to synonymously as 1-(4-ethoxy-4-oxobutan-2-yl) 4-{3-[3-({4-[(4-ethoxy-4-oxobutan-2-yl)oxy]-4-oxobutanoyl}oxy)-2-hydroxypropoxy]-2-hydroxypropyl}-butanedioate.

The diglycerol triester of succinic acid esterified with ethyl 3-hydroxybutanoate may also be referred to synonymously as 1-{3-[2,3-bis({4-[(4-ethoxy-4-oxobutan-2-yl)oxy]-4-oxobutanoyl}oxy)propoxy]-2-hydroxypropyl}-4-(4-ethoxy-4-oxobutan-2-yl)-butanedioate.

The diglycerol tetraester of succinic acid esterified with ethyl 3-hydroxybutanoate may also be synonymously referred to as 1-{1-[2,3-bis({4-[(4-ethoxy-4-oxobutan-2-yl)oxy]-4-oxobutanoyl}oxy)propoxy]-3-({4-[(4-ethoxy-4-oxobutan-2-yl)oxy]-4-oxobutanoyl}oxy)propan-2-yl}-4-(4-ethoxy-4-oxobutan-2-yl)-butanedioate.

Characterization is performed by mass spectrometry (MS), gel permeation chromatography (GPC) and proton resonance spectroscopy (¹H-NMR).

In the following, the course of the reaction is shown schematically. In the case of by-products, free hydroxyl groups may optionally also be esterified (completely or partially) by a radical —C(O)—CH₂—CH(OH)—CH₃ (esterification with ethyl 3-hydroxybutanoate) or a radical —C(O)—CH₂—CH₂—COOH (esterification with succinic acid anhydride):

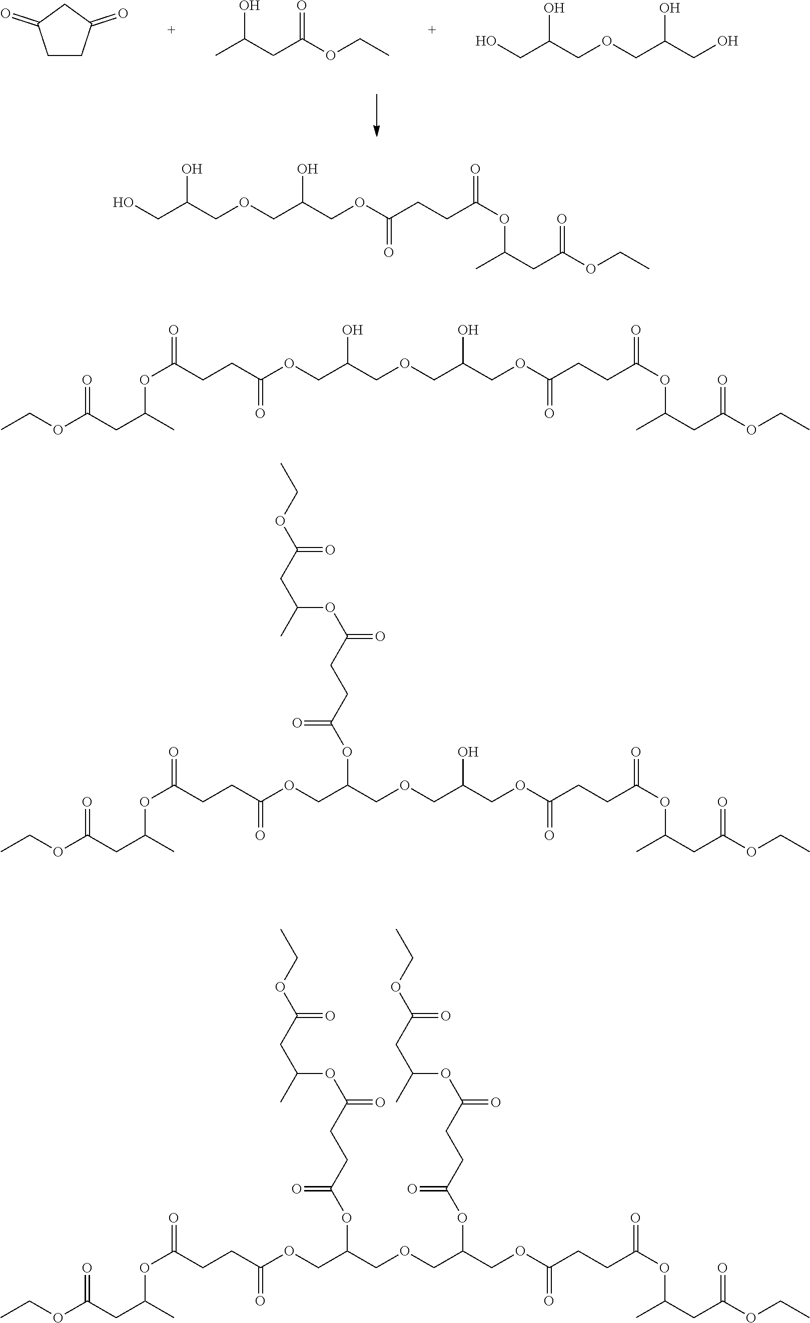

### I. 2. Production of Diglycerol Esters of Succinic Acid Esterified with Ethyl 3-hydroxybutanoate Using the Free Succinic Acid

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic), 118 g succinic acid and 40 g diglycerol are provided. The reaction mixture is reacted with stirring at 120° C. and under N₂ for 7 h, and the resulting reaction water is continuously withdrawn by distillation. Subsequently, excess 3-hydroxybutyric acid ethyl ester and excess succinic acid ester of ethyl 3-hydroxybutanoate are withdrawn by distillation under vacuum. The residue obtained is steam treated in a vacuum for 2 to 4 hours if necessary.

A mixture of mono-, di-, tri- and tetra-valent diglycerol esters (i.e. diglycerol monoester, diglycerol diester, diglycerol triester and diglycerol tetraester) of succinic acid esterified with ethyl 3-hydroxybutanoate is obtained.

By-products formed include very small amounts of diglycerol esters of 3-hydroxybutyric acid and diglycerol esters of succinic acid.

Characterization is performed by mass spectrometry (MS), gel permeation chromatography (GPC) and proton resonance spectroscopy (¹H-NMR).

### I. 3. Production of Diglycerol Esters of Succinic Acid Esterified with Ethyl 3-hydroxybutanoate Using Succinic Acid Diethyl Ester

In a 500-ml-multi-neck flask equipped with a dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic), 174 g succinic acid diethyl ester and 40 g diglycerol are provided. The reaction mixture is reacted with stirring at 120° C. and at N₂ for 7 h, and the resulting ethanol is continuously withdrawn by distillation. Subsequently, excess 3-hydroxybutyric acid ethyl ester and excess succinic acid ester of ethyl 3-hydroxybutanoate are withdrawn by distillation under vacuum. The residue obtained is steam treated in a vacuum for 2 to 4 hours, if necessary.

A mixture of mono-, di-, tri- and tetra-valent diglycerol esters (i.e. diglycerol monoester, diglycerol diester, diglycerol triester and diglycerol tetraester) of succinic acid esterified with ethyl 3-hydroxybutanoate is obtained.

By-products formed include very small amounts of diglycerol esters of 3-hydroxybutyric acid and diglycerol esters of succinic acid.

Characterization is performed by mass spectrometry (MS), gel permeation chromatography (GPC) and proton resonance spectroscopy (¹H-NMR).

### I. 4. Production of Further Diglycerol Esters of Polycarboxylic Acids Esterified with Ethyl 3-hydroxybutanoate

The previously performed synthesis examples according to I. 1., I. 2. and I. 3. are carried out again, however, using the following polycarboxylic acids (first method alternative in each case as free acids) and their respective anhydrides (second method alternative) as well as their respective ethyl esters (third method alternative): tartaric acid, citric acid, malic acid, adipic acid, fumaric acid and maleic acid. Comparable results are obtained in each case.

### II. Examples of Production of a Particular Embodiment of the Method According to the Invention as a Two-Step Synthesis without the Use of a Catalyst

### II. 1. Production of Diglycerol Esters of Succinic Acid Esterified with Ethyl 3-hydroxybutanoate Using Succinic Acid Anhydride

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic) and 100 g succinic acid anhydride are provided. The reaction mixture is allowed to react with stirring at 120° C. and under N₂ for 7 h. At the end of the reaction time, a sample is taken for analysis. Then 40 g of diglycerol are added and the reaction mixture is reacted with stirring at 120° C. and under N₂ for another 7 h, and the resulting water of reaction is withdrawn continuously by distillation. After the reaction time has elapsed, another sample is taken for analysis.

Subsequently, excess 3-hydroxybutyric acid ethyl ester and excess succinic acid ester of ethyl 3-hydroxybutanoate are withdrawn by distillation under vacuum. The residue obtained is steam treated in a vacuum for 2 to 4 hours, if necessary.

As in the case of the one-step synthesis, a mixture of mono-, di-, tri- and tetravalent diglycerol esters (=esters of diglycerol) (i.e. diglycerol monoester, diglycerol diester, diglycerol triester 35 and diglycerol tetraester) of succinic acid esterified with ethyl 3-hydroxybutanoate is obtained.

By-products formed include very small amounts of diglycerol esters of 3-hydroxybutyric acid and diglycerol esters of succinic acid.

Characterization is performed by mass spectrometry (MS), gel permeation chromatography (GPC) and proton resonance spectroscopy (¹H-NMR).

The samples taken at the end of the first and second reaction steps are analyzed by GC (gas chromatography); GC area analyses are summarized in table 1.

TABLE 1
GC area analysis [%] of the reaction of succinic
anhydride, ethyl 3-hydroxybutanoate and diglycerol.

| | after the | after the |
|---|---|---|
| | first step | second step |
| | of the method | of the method |
| succinic acid anhydride | 3.1 | 0.1 |
| succinic acid | 2.2 | 1.1 |
| 3-BBB-EE | 19.2 | 7.5 |
| BS-(BHB-EE) monoester | 65.2 | 44.9 |
| BS-(BHB-EE) diester | 1.3 | 3.6 |
| diglycerin | — | 7.6 |
| PG(2)-(BS-(BHB-EE)) monoester | — | 12.6 |
| PG(2)-(BS-(BHB-EE)) monoester | — | 6.7 |
| PG(2)-(BS-(BHB-EE)) monoester | — | 6.2 |
| by-products * | 0.8 | 6.5 |
| unknown | 8.2 | 3.2 |

* including monoethyl succinate, BHB dimer, BHB diglycerol esters, diglycerol esters of 3-hydroxybutyric acid and diglycerol esters of succinic acid

In the following the course of the reaction is shown schematically (in the case of by-products free hydroxyl groups may optionally also be esterified, completely or partially, by a radical —C(O)—CH₂—CH(OH)—CH₃ (esterification with ethyl 3-hydroxybutanoate) or a radical —C(O)—CH₂—CH₂—COOH (esterification with succinic acid anhydride)):

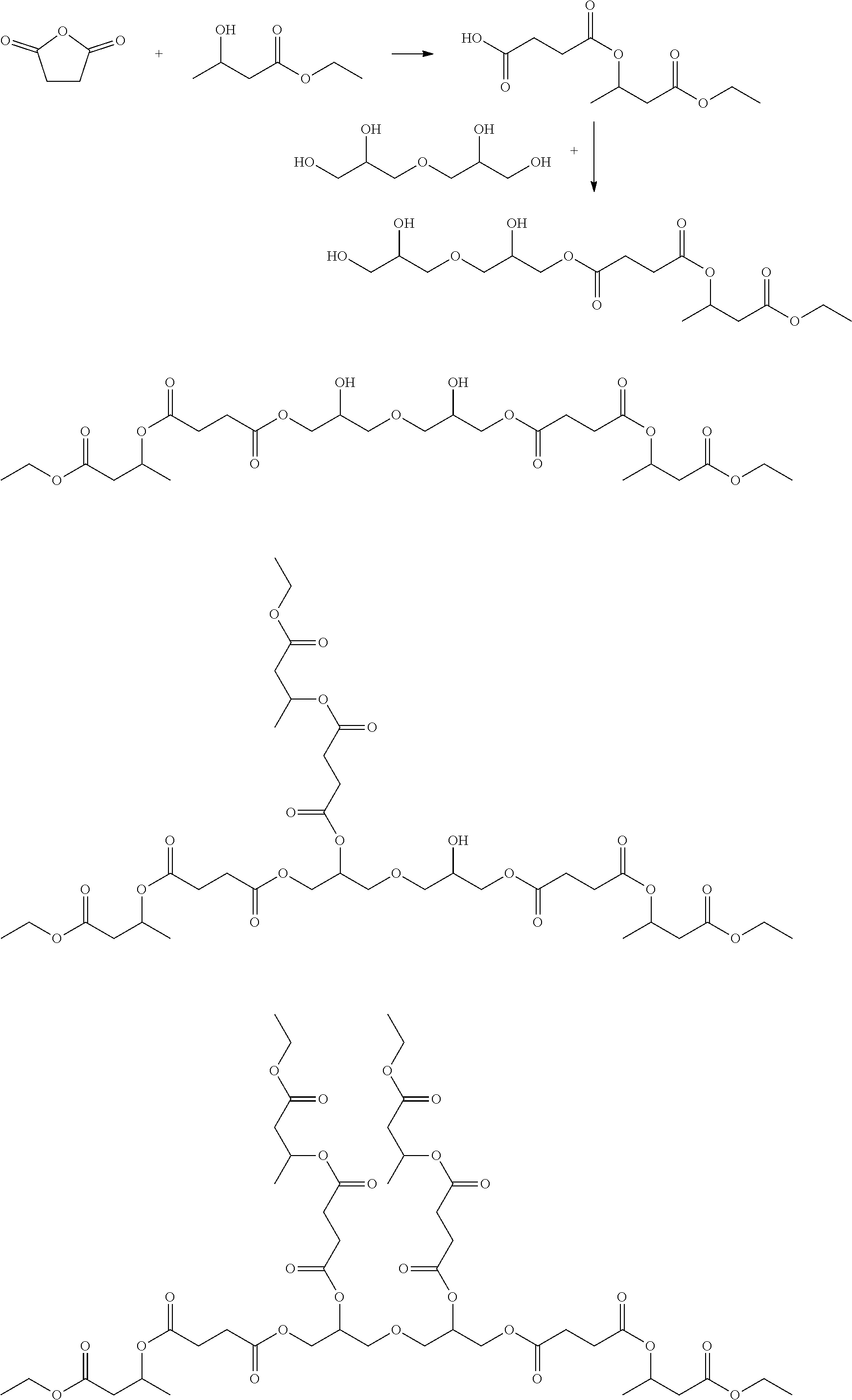

### II. 2. Production of Diglycerol Esters of Succinic Acid Esterified with Ethyl 3-hydroxybutanoate Using the Free Succinic Acid

In a 500-ml-multi-neck flask with dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic) and 118 g succinic acid are provided. The reaction mixture is reacted with stirring at 120° C. and under N₂ for 7 h and the resulting reaction water is continuously withdrawn by distillation. Then 40 g of diglycerol are added and the reaction mixture is reacted with stirring at 120° C. and under N₂ for another 7 h and the resulting reaction water is continuously withdrawn by distillation.

Subsequently, excess 3-hydroxybutyric acid ethyl ester and excess succinic acid ester of ethyl 3-hydroxybutanoate are withdrawn by distillation under vacuum. The residue obtained is steam treated in a vacuum for 2 to 4 hours, if necessary.

A mixture of mono-, di-, tri- and tetra-valent diglycerol esters (i.e. diglycerol monoester, diglycerol diester, diglycerol triester and diglycerol tetraester) of succinic acid esterified with ethyl 3-hydroxybutanoate is obtained.

By-products formed include very small amounts of diglycerol esters of 3-hydroxybutyric acid and diglycerol esters of succinic acid.

Characterization is performed by mass spectrometry (MS), gel permeation chromatography (GPC) and proton resonance spectroscopy (¹H-NMR).

### II. 3. Production of Diglycerol Esters of Succinic Acid Esterified with Ethyl 3-hydroxybutanoate Using Succinic Acid Diethyl Ester

In a 500-ml-multi-neck flask equipped with a dephlegmator (partial condenser) and distillation bridge, 132 g (R)/(S)-3-hydroxybutyric acid ethyl ester (3-BHB-EE=ethyl 3-hydroxybutanoate or 4-ethoxy-4-oxobutan-2-ol) (racemic) and 174 g succinic acid diethyl ester are provided. The reaction mixture is reacted with stirring at 120° C. and under N₂ for 7 h, and the resulting ethanol is continuously withdrawn by distillation. Then 40 g diglycerol are added and the reaction mixture is reacted with stirring at 120° C. and under N₂ for another 7 h and the resulting ethanol is continuously withdrawn by distillation.

Subsequently, excess 3-hydroxybutyric acid ethyl ester and excess succinic acid ester of ethyl 3-hydroxybutanoate are withdrawn by distillation under vacuum. The residue obtained is steam treated in a vacuum for 2 to 4 hours, if necessary.

A mixture of mono-, di-, tri- and tetra-valent diglycerol esters (i.e. diglycerol monoester, diglycerol diester, diglycerol triester and diglycerol tetraester) of succinic acid esterified with ethyl 3-hydroxybutanoate is obtained.

By-products formed include very small amounts of diglycerol esters of 3-hydroxybutyric acid and diglycerol esters of succinic acid.

Characterization is performed by mass spectrometry (MS), gel permeation chromatography (GPC) and proton resonance spectroscopy (¹H-NMR).

### II. 4. Production of Further Diglycerol Esters of Polycarboxylic Acids Esterified with Ethyl 3-hydroxybutanoate

The previously performed synthesis examples according to II. 1., II. 2. and II. 3. are carried out again, however, using the following polycarboxylic acids and their respective anhydrides as well as ethyl esters: tartaric acid, citric acid, malic acid, adipic acid, fumaric acid and maleic acid. Comparable results are obtained.

### III. Production Examples of an Alternative Particular Embodiment of the Method According to the Invention Using a Metal Catalyst

All chemical synthesis examples previously described in section I. and section II. are carried out again, however, with the addition of titanium tetrabutylate as a catalyst (titanium(IV)-catalyst). The titanium(IV)-catalyst is provided into the flask together with the other reactants. Subsequently, the reaction method corresponds to the examples described above. Comparable results are obtained. The catalyst is separated and recycled at the end of the reaction.

### IV. Production Examples of a Further Alternative Particular Embodiment of the Method According to the Invention Using an Enzyme Catalyst

All chemical synthesis examples previously described in section I. and section II. are performed again, however, with the addition of an immobilized enzyme (CALB lipase on polymer support, derived from Candida antarctica, e.g. Novozym® 435 from Sigma-Aldrich or Merck or Lipozym® 435 from Strem Chemicals, Inc.) as catalyst and at 70° C. under vacuum. Comparable results are obtained. The enzyme is separated and recycled after the end of the reaction.

### V. Physiological Application Tests: In Vitro Digestion Tests

### V. 1. Digestion Experiments (Splitting or Cleavage Experiments) of Carboxylic Acid Esters of 3-hydroxybutanoate According to the Invention

By means of cleavage experiments, it is shown that polyglycerol esters of polycarboxylic acids esterified with ethyl 3-hydroxybutanoate or mixtures thereof prepared according to the invention (cf. previously described experiments according to I., II. III and IV.), including the reaction by-products, can be cleaved in the human gastrointestinal tract.

In each case, purified reaction products obtained by the method according to the invention are used as starting mixtures:
diglycerol ester mixture of tartaric acid esterified with ethyl 3-hydroxybutanoate,
diglycerol ester mixture of citric acid esterified with ethyl 3-hydroxybutanoate,
diglycerol ester mixture of malic acid esterified with ethyl 3-hydroxybutanoate,
diglycerol ester mixture of adipic acid esterified with ethyl 3-hydroxybutanoate,
diglycerol ester mixture of fumaric acid esterified with ethyl 3-hydroxybutanoate and
diglycerol ester mixture of maleic acid esterified with ethyl 3-hydroxybutanoate.

For the cleavage experiments under near-body conditions two media are investigated:
FaSSGF, which simulates the stomach
FaSSIF, which simulates the intestinal tract.

Both media are from the company Biorelevantm, Ltd. in Great Britain. In addition, in some experiments porcine pancreas is added (Panzytrat® 40,000, Fa. Allergan).

The results of the cleavage experiments in a FaSSGF or FaSSIF medium with Panzytrat® and without Panzytrat® (both 35° C., 24 h) show that the samples hydrolyze under FaSSGF conditions with Panzytrat® and without Panzytrat®; this is mainly due to the low pH value (pH=1.6) of the medium. Under FaSSIF conditions, a lower conversion using Panzytrat® takes place.

In all cleavage experiments, it can be seen that the cleavage proceeds in the form of a cascade (i.e. the diglycerol tetraester becomes the diglycerol triester, the diglycerol triester becomes the diglycerol diester, etc.). Furthermore, the ethyl 3-hydroxybutanoate is cleaved from the polycarboxylic acid and then split into the free butyric acid and ethanol. In total, the polycarboxylic acids and the free butyric acid that can be utilized by the body are thus released, and furthermore the non-toxic, physiologically compatible carrier diglycerol is released, which is excreted by the body.

Overall, therefore, there is a retardation effect (i.e. the release of 3-hydroxybutyric acid and polycarboxylic acid are delayed and continuous, respectively, over a longer period of time).

### V. 2. Further Digestion Experiments (Cleavage Experiments) of Carboxylic Acids of 3-hydroxybutanoate According to the Invention

Cleavage Experiments with Pancreatin

2 g of a diglycerol ester of polycarboxylic acids esterified with ethyl 3-hydroxybutanoate prepared as described above or a mixture thereof (mixture of corresponding diglycerol mono-, diglycerol di-, diglycerol tri- and/or diglycerol tetraesters of polycarboxylic acids esterified with 3-hydroxybutanoate) are dissolved in 50 g of water and 0.5 g (1% by weight) of pancreatin is added. The pancreatin is used in the form of the commercially available product Panzytrat® 40,000 from Allergan. The whole mixture is stirred on a hot plate at 50° C.; the course of the reaction is determined and followed by continuous recording of the acid number over time. The acid number increases over the observation period (cleavage of diglycerol esters of polycarboxylic acids esterified with 3-hydroxybutanoate). The conversion/time course of the aqueous cleavage of the diglycerol esters of polycarboxylic acids esterified with 3-hydroxybutanoate according to the invention by means of pancreatin, including the increase of the acid number over time, proves the desired decomposition of the reactant mixture to the free polycarboxylic acid and the free 3-hydroxybutyric acid. This is confirmed by corresponding analytics. The experiment proves that the starting mixture according to the invention is a suitable physiological precursor for 3-hydroxybutyric acid or its esters (3-hydroxybutanoates) for the corresponding keto-body therapies.

### V. 3. Conclusions

The cleavage experiments described above demonstrate that the diglycerol esters of polycarboxylic acids esterified with 3-hydroxybutanoate are efficient precursors or metabolites of free hydroxybutyric acid or its esters (here: ethyl esters), particularly with regard to their intended effect, which are present in physiologically tolerable or physiologically compatible form. Likewise, metabolically utilizable or convertible polycarboxylic acids occurring in the natural metabolism (e.g. citrate cycle) or their derivatives, especially salts, are formed (e.g. citric acid or citrates, malic acid or malates, tartaric acid or tartrates, etc.). The diglycerol serves as a physiologically compatible, non-toxic carrier molecule for the bonding of a large number of active ingredient molecules (=polycarboxylic acids esterified with 3-hydroxybutanoate), which can itself be readily excreted; this results in a high active ingredient density on the one hand and a desired controlled, especially sustained, release on the other.

### VI. Further Testing (Organoleptic and Toxicity)

Further experiments and test series are carried out with respect to organoleptic and toxicity of the diglycerol esters of polycarboxylic acids esterified with 3-hydroxybutanoate according to the invention. These show that the diglycerol esters of polycarboxylic acids esterified with 3-hydroxybutanoate according to the invention are organoleptically acceptable and compatible, especially exhibit significantly improved organoleptic properties compared with pure 3-hydroxybutyric acid as well as its salts and esters, and moreover do not exhibit any toxicity contrary to the application.

1-81. (canceled)

82. A method for producing polyglycerol esters of polycarboxylic acids esterified with oxobutanol,
wherein the following reactants (i), (ii) and (iii) are reacted with one another:
(i) at least one oxobutanol of the general formula (I)
CH₃—CH(OH)—CH₂—C(O)OR¹ (I)
wherein, in the general formula (I), the radical R¹ is selected among C₁-C₅-alkyl and hydroxy-C₃-C₅-alkyl,
(ii) at least one polycarboxylic acid (II) selected among the polycarboxylic acid (II) in its free form and in the form of its salts, esters and anhydrides,
(iii) at least one polyglycerol (III);
wherein the method is carried out as one of a one-step method and a method comprising several steps, and
wherein the method is carried out in the absence of any solvent;
wherein, when the polycarboxylic acid (II) is used in the form of a free acid, water is formed simultaneously during the reaction, wherein the water is continuously withdrawn from the reaction;
wherein, when the polycarboxylic acid (II) is used in the form of an anhydride, the corresponding free polycarboxylic acid (II) and water are formed, wherein the resulting free polycarboxylic acid (II) is further reacted or, after the reaction has taken place, is removed and recycled and wherein the water is continuously withdrawn from the reaction;
wherein, when the polycarboxylic acid (II) is used in the form of an ester, the corresponding ester alcohol and water are formed, wherein the resulting ester alcohol and water are continuously withdrawn from the reaction;
so that, as a reaction product (IV), one or more polyglycerol esters of polycarboxylic acids esterified with oxobutanol are obtained.

83. The method according to claim 82,
wherein the method is carried out in at least two steps,
wherein:
(a) in a first method step (a), the at least one oxobutanol of the general formula (I) is reacted with the at least one polycarboxylic acid (II) in an esterification reaction,
so that, as a reaction intermediate (IV′) of method step (a), one or more polycarboxylic acid esters of oxobutanol are obtained; and
(b) subsequently, in a second method step (b), the reaction intermediate product (IV′) obtained in method step (a) is reacted with the at least one polyglycerol (III) in an esterification reaction.

84. The method according to claim 82,
wherein the polycarboxylic acid (II) is selected from the group consisting of succinic acid, tartaric acid, citric acid, malic acid, adipic acid, fumaric acid and maleic acid.

85. The method according to claim 82,
wherein the polycarboxylic acid (II) is selected among naturally occurring carboxylic acids and their anhydrides and derivatives occurring in one of human and animal metabolism.

86. The method according to claim 82,
wherein the polycarboxylic acid (II) is an ingredient approved under food law.

87. The method according to claim 82,
wherein the polyglycerol corresponds to the general formula (IIIa)
HO—CH₂—CH(OH)—CH₂—[O—CH₂—CH(OH)—CH₂]ₚ—OH (IIIa)
wherein, in general formula (IIIa), the variable p represents an integer from 1 to 6.

88. The method according to claim 82,
wherein the polyglycerol (III) is a diglycerol of formula (IIIb)
HO—CH₂—CH(OH)—CH₂—O—CH₂—CH(OH)—CH₂—OH (IIIb).

89. The method according to claim 82,
wherein the polyglycerol (III) is not propane-1,2,3-triol (glycerol).

90. The method according to claim 82,
wherein hydroxyl groups and carboxyl groups still present in the reaction product after the reaction has been performed are at least partially functionalized.

91. The method according to claim 82,
wherein the reaction is followed by an at least partial esterification of hydroxyl groups and carboxyl groups still present after the reaction has been performed.

92. A reaction intermediate product in the form of a polycarboxylic acid ester of oxobutanol,
wherein the reaction intermediate product is obtainable as the reaction intermediate product (IV′) of the first method step (a) of the method according to claim 83.

93. A reaction product in the form of a polyglycerol ester of polycarboxylic acids esterified with oxobutanol,
wherein the reaction product is obtainable by the method according to claim 82.

94. A polyglycerol ester of polycarboxylic acids esterified with oxobutanol,
wherein the polyglycerol ester of polycarboxylic acids esterified with oxobutanol corresponds to the general formula (IVa)
R⁴O—CH₂—CH(OR⁴)—CH₂—[O—CH₂—CH(OR⁴)—CH₂]ₚ—OR⁴ (IVa)
wherein, in the general formula (IVa), the radical R⁴, independently of one another, represents:
hydrogen;
a radical R¹—O—C(O)—CH₂—CH(CH₃)—O—(O)C—X—C(O)—,
wherein the radical R¹ is selected among C₁-C₅-alkyl and hydroxy-C₃-C₅-alkyl, and
wherein X represents a saturated or unsaturated organic radical comprising 1 to 10 carbon atoms, which may be mono- or polysubstituted with one or more of the following radicals: hydroxyl, carboxyl, —O—C(O)—CH₂—CH(OH)—CH₃ and —C(O)—O—CH(CH₃)—CH₂—C(O)OR¹ with R¹ as defined hereinbefore;
a radical H₃C—CH(OH)—CH₂—C(O)—;
a radical HOOC—X—C(O)— with X as defined hereinbefore;
however, with the proviso that at least one radical R⁴ represents a radical R¹—O—C(O)—CH₂—CH(CH₃)—O—(O)C—X—C(O)— as defined hereinbefore; and
wherein, in the general formula (IVa), the variable p represents an integer from 1 to 6.

95. The polyglycerol ester of polycarboxylic acids esterified with oxobutanol according to claim 94,
wherein the polyglycerol ester of polycarboxylic acids esterified with oxobutanol corresponds to the general formula (IVa)
R⁴O—CH₂—CH(OR⁴)—CH₂—[O—CH₂—CH(OR⁴)—CH₂]ₚ—OR⁴ (IVa)
wherein, in the general formula (IVa), the radical R*, independently of one another, represents:
hydrogen;
a radical R¹—O—C(O)—CH₂—CH(CH₃)—O—(O)C—X—C(O)—,
wherein the radical R¹ is selected among C₁-C₅-alkyl and hydroxy-C₃-C₅-alkyl, and
wherein X represents a saturated or unsaturated organic radical comprising 1 to 10 carbon atoms, which may be mono- or polysubstituted with one or more of the following radicals: hydroxyl, carboxyl, —O—C(O)—CH₂—CH(OH)—CH₃ and —C(O)—O—CH(CH₃)—CH₂—C(O)OR¹ with R¹ as defined hereinbefore;
however, with the proviso that at least one radical R¹ represents a radical R¹—O—C(O)—CH₂—CH(CH₃)—O—(O)C—X—C(O)— as defined hereinbefore; and
wherein, in the general formula (IVa), the variable p represents an integer from 1 to 6.

96. The polyglycerol ester of polycarboxylic acids esterified with oxobutanol according to claim 94,
wherein the polyglycerol ester of polycarboxylic acids esterified with oxobutanol corresponds to the general formula (IVa)
R¹O—CH₂—CH(OR⁴)—CH₂—[O—CH₂—CH(OR⁴)—CH₂]ₚ—OR⁴ (IVa)
wherein, in the general formula (IVa), the radical R⁴, independently of one another, represents:
hydrogen;
a radical R¹—O—C(O)—CH₂—CH(CH₃)—O—(O)C—X—C(O)—,
wherein the radical R¹ is selected among C₁-C₅-alkyl and hydroxy-C₃-C₅-alkyl, and
wherein X represents a saturated or unsaturated organic radical comprising 1 to 10 carbon atoms, which is mono- or polysubstituted with one or more of the following radicals: hydroxyl, carboxyl, —O—C(O)—CH₂—CH(OH)—CH₃ and —C(O)—O—CH(CH₃)—CH₂—C(O)OR¹ with R¹ as defined hereinbefore;
however, with the proviso that at least one radical R¹ represents a radical R¹—O—C(O)—CH₂—CH(CH₃)—O—(O)C—X—C(O)—, as defined hereinbefore; and
wherein, in the general formula (IVa), the variable p represents an integer from 1 to 6.

97. The polyglycerol ester of polycarboxylic acids esterified with oxobutanol according to claim 94,
wherein the polyglycerol ester of polycarboxylic acids esterified with oxobutanol corresponds to the general formula (IVb)
R⁵O—CH₂—CH(OR⁵)—CH₂—[O—CH₂—CH(OR⁵)—CH₂]ₚ—OR⁵ (IVb)
wherein, in the general formula (IVb), the radical R⁵, independently of one another, represents:
hydrogen;
a radical R¹—O—C(O)—CH₂—CH(CH₃)—O—Y—,
wherein the radical R¹ is selected among C₁-C₅-alkyl and hydroxy-C₃-C₅-alkyl, and
wherein Y is derived from a polycarboxylic acid selected from the group consisting of succinic acid, tartaric acid, citric acid, malic acid, adipic acid, fumaric acid and maleic acid, especially wherein in the case of citric acid the further carboxyl group present is esterified with a radical —CH(CH₃)—CH₂—C(O)OR¹ with R¹ as defined hereinbefore,
a radical H₃C—CH(OH)—CH₂—C(O)—;
a radical —Y—OH with Y as defined hereinbefore;
however, with the proviso that at least one radical R⁵ represents a radical R¹—O—C(O)—CH₂—CH(CH₃)—O—Y— with R¹ and Y each as defined hereinbefore; and
wherein, in the general formula (IVb), the variable p represents an integer from 1 to 6.

98. The polyglycerol ester of polycarboxylic acids esterified with oxobutanol according to claim 94,
wherein the polyglycerol ester of polycarboxylic acids esterified with oxobutanol corresponds to the general formula (IVb)
R⁵O—CH₂—CH(OR⁵)—CH₂—[O—CH₂—CH(OR⁵)—CH₂]ₚ—OR⁵ (IVb)
wherein, in the general formula (IVb), the radical R⁵, independently of one another, represents:
hydrogen;
a radical R¹—O—C(O)—CH₂—CH(CH₃)—O—Y—,
wherein the radical R¹ is selected among C₁-C₅-alkyl and hydroxy-C₃-C₅-alkyl, and
wherein Y represents one of the following radicals: 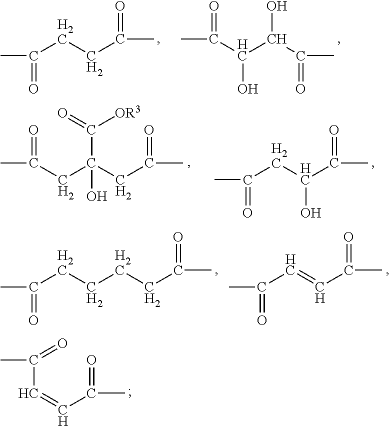
wherein, in the radicals, the radical R³ is selected among hydrogen and a radical —CH(CH₃)—CH₂—C(O)OR¹ with R¹ as defined hereinbefore;
a radical H₃C—CH(OH)—CH₂—C(O)—;
a radical Y—OH with Y as defined hereinbefore;
however, with the proviso that at least one radical R⁵ represents a radical R¹—O—C(O)—CH₂—CH(CH₃)—O—Y— with R¹ and Y each as defined hereinbefore; and
wherein, in the general formula (IVb), the variable p represents an integer from 1 to 6.

99. The polyglycerol ester of polycarboxylic acids esterified with oxobutanol according to claim 94,
wherein the polyglycerol ester of polycarboxylic acids esterified with oxobutanol corresponds to the general formula (IVc)
R⁴O—CH₂—CH(OR⁴)—CH₂—O—CH₂—CH(OR⁴)—CH₂—OR⁴ (IVc)
wherein, in the general formula (IVc), the radical R⁴, independently of one another, represents
hydrogen;
a radical R¹—O—C(O)—CH₂—CH(CH₃)—O—(O)C—X—C(O)—,
wherein the radical R¹ is selected among C₁-C₅-alkyl and hydroxy-C₃-C₅-alkyl, and
wherein X represents a saturated or unsaturated organic radical comprising 1 to 10 carbon atoms, which may be mono- or polysubstituted with one or more of the following radicals: hydroxyl, carboxyl, —O—C(O)—CH₂—CH(OH)—CH₃ and —C(O)—O—CH(CH₃)—CH₂—C(O)OR¹ with R¹ as defined hereinbefore;
a radical H₃C—CH(OH)—CH₂—C(O)—;
a radical HOOC—X—C(O)— with X as defined hereinbefore;
however, with the proviso that at least one radical R¹ represents a radical R¹—O—C(O)—CH₂—CH(CH₃)—O—(O)C—X—C(O)— as defined hereinbefore.

100. The polyglycerol ester of polycarboxylic acids esterified with oxobutanol according to claim 94,
wherein the polyglycerol ester of polycarboxylic acids esterified with oxobutanol corresponds to the general formula (IVd)
R⁵O—CH₂—CH(OR⁵)—CH₂—O—CH₂—CH(OR⁵)—CH₂—OR⁵ (IVd)
wherein, in the general formula (IVd), the radical R⁵, independently of one another, represents
hydrogen;
a radical R¹—O—C(O)—CH₂—CH(CH₃)—O—Y—,
wherein the radical R¹ is selected among C₁-C₅-alkyl and hydroxy-C₃-C₅-alkyl, and
wherein Y represents one of the following radicals: 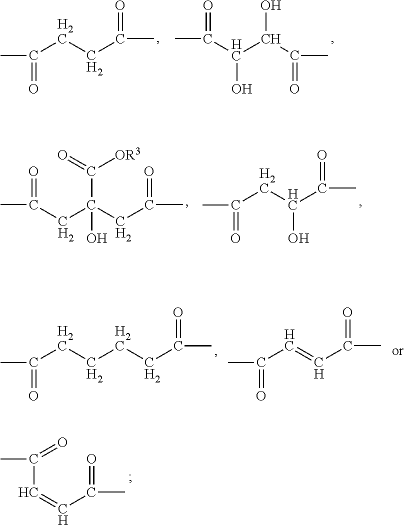
wherein, in the radicals, the radical R³ represents hydrogen or a radical —CH(CH₃)—CH₂—C(O)OR¹ with R¹ as defined hereinbefore;
a radical H₃C—CH(OH)—CH₂—C(O)—;
a radical Y—OH with Y as defined hereinbefore;
however, with the proviso that at least one radical R⁵ represents a radical R¹—O—C(O)—CH₂—CH(CH₃)—O—Y— with R¹ and Y each as defined hereinbefore.

101. A mixture comprising at least two different polyglycerol esters of polycarboxylic acids esterified with oxobutanol, as defined in claim 94.

102. A pharmaceutical composition comprising at least one polyglycerol ester of polycarboxylic acids esterified with oxobutanol,
wherein the polyglycerol ester of polycarboxylic acids esterified with oxobutanol corresponds to the general formula (IVa)
R⁴O—CH₂—CH(OR⁴)—CH₂—[O—CH₂—CH(OR⁴)—CH₂]ₚ—OR⁴ (IVa)
wherein, in the general formula (IVa), the radical R⁴, independently of one another, represents:
hydrogen;
a radical R¹—O—C(O)—CH₂—CH(CH₃)—O—(O)C—X—C(O)—,
wherein the radical R¹ is selected among C₁-C₅-alkyl and hydroxy-C₃-C₅-alkyl, and
wherein X represents a saturated or unsaturated organic radical comprising 1 to 10 carbon atoms, which may be mono- or polysubstituted with one or more of the following radicals: hydroxyl, carboxyl, —O—C(O)—CH₂—CH(OH)—CH₃ and —C(O)—O—CH(CH₃)—CH₂—C(O)OR¹ with R¹ as defined hereinbefore;
a radical H₃C—CH(OH)—CH₂—C(O)—;
a radical HOOC—X—C(O)— with X as defined hereinbefore;
however, with the proviso that at least one radical R⁴ represents a radical R¹—O—C(O)—CH₂—CH(CH₃)—O—(O)C—X—C(O)— as defined hereinbefore; and
wherein, in the general formula (IVa), the variable p represents an integer from 1 to 6.

103. The pharmaceutical composition according to claim 102,
wherein the pharmaceutical composition is a drug or a medicament.

104. A method of treating a human or an animal suffering from a disease of the human or animal body,
wherein the method comprises the step of administering to said human or animal a therapeutically efficient amount of at least one polyglycerol ester of polycarboxylic acids esterified with oxobutanol,
wherein the polyglycerol ester of polycarboxylic acids esterified with oxobutanol corresponds to the general formula (IVa)
R⁴O—CH₂—CH(OR⁴)—CH₂—[O—CH₂—CH(OR⁴)—CH₂]ₚ—OR⁴ (IVa)
wherein, in the general formula (IVa), the radical R*, independently of one another, represents:
hydrogen;
a radical R¹—O—C(O)—CH₂—CH(CH₃)—O—(O)C—X—C(O)—,
wherein the radical R¹ is selected among C₁-C₅-alkyl and hydroxy-C₃-C₅-alkyl, and
wherein X represents a saturated or unsaturated organic radical comprising 1 to 10 carbon atoms, which may be mono- or polysubstituted with one or more of the following radicals: hydroxyl, carboxyl, —O—C(O)—CH₂—CH(OH)—CH₃ and —C(O)—O—CH(CH₃)—CH₂—C(O)OR¹ with R¹ as defined hereinbefore;
a radical H₃C—CH(OH)—CH₂—C(O)—;
a radical HOOC—X—C(O)— with X as defined hereinbefore;
however, with the proviso that at least one radical R⁴ represents a radical R¹—O—C(O)—CH₂—CH(CH₃)—O—(O)C—X—C(O)— as defined hereinbefore; and
wherein, in the general formula (IVa), the variable p represents an integer from 1 to 6.

105. The method according to claim 104,
wherein the disease is selected among diseases associated with a disorder of human or animal energy metabolism.

106. The method according to claim 104,
wherein the disease is selected among diseases associated with a disorder of human or animal keto-body metabolism.

107. A food product comprising at least one polyglycerol ester of polycarboxylic acids esterified with oxobutanol,
wherein the polyglycerol ester of polycarboxylic acids esterified with oxobutanol corresponds to the general formula (IVa)
R⁴O—CH₂—CH(OR⁴)—CH₂—[O—CH₂—CH(OR⁴)—CH₂]ₚ—OR⁴ (IVa)
wherein, in the general formula (IVa), the radical R⁴, independently of one another, represents:
hydrogen;
a radical R¹—O—C(O)—CH₂—CH(CH₃)—O—(O)C—X—C(O)—,
wherein the radical R¹ is selected among C₁-C₅-alkyl and hydroxy-C₃-C₅-alkyl, and
wherein X represents a saturated or unsaturated organic radical comprising 1 to 10 carbon atoms, which may be mono- or polysubstituted with one or more of the following radicals: hydroxyl, carboxyl, —O—C(O)—CH₂—CH(OH)—CH₃ and —C(O)—O—CH(CH₃)—CH₂—C(O)OR¹ with R¹ as defined hereinbefore;
a radical H₃C—CH(OH)—CH₂—C(O)—;
a radical HOOC—X—C(O)— with X as defined hereinbefore;
however, with the proviso that at least one radical R⁴ represents a radical R¹—O—C(O)—CH₂—CH(CH₃)—O—(O)C—X—C(O)— as defined hereinbefore; and
wherein, in the general formula (IVa), the variable p represents an integer from 1 to 6.

108. The food product according to claim 107,
wherein the food product is selected from the group consisting of a food, a dietary supplement, a functional food, a novel food, a food additive, a food supplement, a dietary food, a power snack, an appetite suppressant and a strength sports supplement and an endurance sports supplement.
